(19) United States
(12) Patent Application Publication
(10) Pub. No.: US 2006/0105025 A1
(43) Pub. Date: May 18, 2006

(54) Recovery pet chews

(76) Inventors: Ira D. Hill, Austin, TX (US); Dale G. Brown, Wharton, TX (US); Robert C. Lepple, Missouri City, TX (US); Ronald H. Butler, Scottsdale, AZ (US)

Correspondence Address: BANNER & WITCOFF, LTD., 28 STATE STREET, 28th FLOOR, BOSTON, MA, 02109-9601, US

(21) Appl. No.: 10/989,164

(22) Filed: Nov. 15, 2004

(51) Int. Cl. A61K 36/8998; A61K 36/86; A23K 1/165; A23K 1/17; A61K 36/899; A61K 36/87

(52) U.S. Cl. 424/442; 424/766; 424/750

(57) ABSTRACT

Extruded and densified recovery, ingestible pet chews, comprising a blend of an emulsion, starch, protein, plasticizing substance and water, which indicate recovery, resistance to fracturing and resistance to crumbling properties over the chew-life of the pet chews.

### FIELD OF THE INVENTION

The present invention is directed to extruded and densified recovery, ingestible pet chews comprising a blend of an emulsion, starch, protein, a plasticizing substance and water, which indicate: recovery, resistance to fracturing and resistance to crumbling properties over the chew-life of the pet chew. These pet chews, when certain oral care ingredients are added, are particularly suitable for controlling, disrupting and removing biofilms that are attached to the pet's teeth. As the pet oral care versions of these chews are consumed, they physically remove biofilms while simultaneously releasing substantive, biofilm-disrupting and controlling ingredients throughout the oral cavity of the pet. When certain therapeutic ingredients are added, these pet chews are particularly suitable vehicles for routinely delivering ingestible, therapeutic substances to the pet.

The recovery pet chews of the invention indicate: (a) physical penetration values between about 600 and about 1000 psi, (b) a Shore-D Hardness from between about 25 and 35, (c) recovery value of at least 30%, i.e., the percent recovery two minutes after the chew is penetrated with a standard recovery probe. Recovery values of at least about 30% are preferred.

### BACKGROUND OF THE INVENTION

Unlike humans, pets do not chew and thoroughly masticate their food before swallowing. Because most of their teeth are sectorial in design (i.e., built for tearing and shredding meat from a carcass and not grinding), pets do not have serious problems with caries. That is, due to limited masticating, pets generally do not pack food and debris between their teeth like humans do. However, pets are domesticated carnivores, taken out of the wild. Accordingly, they no longer have the opportunity to regularly rip, tear and/or shred meat from the carcass of their prey. This lack of “carnivore-type” chewing activity renders most domesticated pets vulnerable to gum disease.

Unfortunately, like their civilized owners, domesticated pets generally suffer from gum disease at about the same incidence as adult humans. For example, more than 86% of the dogs and cats older than four years of age that are brought to veterinary clinics have periodontal disease. See Colmery B., Front R., Vet. Clin. N. America, 18:891 (1982).

Periodontal disease is the overwhelming reason for tooth loss in dogs. Unfortunately, in most cases, treatment for periodontal disease must continue for the life of the pet, because of the pet's continued susceptibility and the chronicity of the disease. Groe T. K., The Compendium on Continuing Education, Vol. 564, No. 7, June 1982.

Periodontal disease in the domestic cat was studied by Rerchart P A, et al., and reported in J. Periodent. Res., 19:67 (1984). Periodontal disease is the most common dental disease in cats. J.V.D., Vol. 5, No. 2, June 1988, and is the most prevalent disease condition found in cats today, Cats Magazine, 16-18, January 1987.

Periodontal problems are progressive. The first occurrence is the formation of plaque (more accurately described as biofilm), which is a transparent, adhesive fluid composed of the mucin in saliva, food particles, sloughed epithelial cells from the abrasive process of eating and the mouth's resident bacteria (usually aerobic Gram-positive, nonmotile cocci). The adhesive matrix that contains the bacteria is called the pellicle. Soft plaque can be removed from teeth by the mechanical action of brushing. If plaque is not removed, the mineral salts in the saliva, particularly calcium salts, will precipitate into the plaque forming hard dental calculus (tartar).

Dental calculus, or tartar, is recognized as a recurring calcified deposit on the surfaces of the teeth of domestic animals, including dogs and cats, as well as humans. It is generally recognized that dental calculus develops in a sequential process that involves the accumulation of dental plaque and the subsequent calcification of the plaque by saliva, which has very high concentrations of calcium and phosphate. Although calculus, per se, is not directly responsible for the development of oral diseases, it is recognized as a secondary, or contributing, factor in the development of periodontal disease because: (1) its presence on the teeth serves as a local irritant to the adjacent soft tissues, eliciting an inflammatory response (and soft tissue inflammation is the initial phase of periodontal disease); (2) it interferes with the normal cleansing of tooth surfaces, which occurs during the mastication of food or through the performance of conventional oral hygiene procedures, such as toothbrushing; and (3) it harbors bacterial toxins, which exacerbate periodontal disease formation, by virtue of its porosity. Once formed, calculus deposits can only be removed through concerted mechanical procedures, i.e., a dental prophylaxis. Thus, the prevention of dental calculus is of importance not only for cosmetic reasons, but also because of dental calculus' secondary role in the development of periodontal disease, and the resultant systemic infections, alveolar bone recession, tooth loss and the most owner recognizable symptom, adverse mouth/breath odors.

At present, the recognized approaches for the prevention of dental calculus formation are: (1) the meticulous, daily removal of dental plaque prior to its calcification; (2) the daily application of crystal growth inhibitors that interfere with the calcification of dental plaque by saliva; and (3) the daily application of sequestering agents such as described in U.S. Pat. No. 5,296,217. Known crystal growth inhibitors include various soluble pyrophosphates, sodium tripolyphosphate, soluble diphosphonates, and certain soluble zinc compounds, such as zinc chloride. These crystal growth inhibitors are currently being used in dentifrices and mouthwashes for preventing dental calculus formation in humans. Soluble pyrophosphates are also currently being cooked or baked in the dough of commercially-prepared diets for dogs and cats for the stated purpose of presenting dental calculus formation in these domestic animals.

When tartar or plaque (biofilm) collects on the teeth, it creates pressure on the gums causing them to become inflamed and to recede. Affected gums appear reddish-blue in color and bleed easily. Teeth in neglected pets may become loose. At this state, pus can be expressed from the surface of the gums when mild pressure is applied. Stoder E. and Stapley R. D., Veterinary Medicine/Small Animal Clinician, 1124, October 1973.

The organisms present in inflamed gum tissue are usually anaerobic, Gram-negative, motile bacilli. See: Eisner E. R., Veterinary Medicine, 97-104, January 1989, Frost R., Williams C. A., Vet. Clin. N. Amer., 16(5):851-874 (1986); Harvey E. E., et al., Textbook of Veterinary Internal Medicine, W.B. Saunders, Philadelphia, Pa. (1982), pp. 1126-1187. Harvey C. E., Veterinary Dentistry, W.B. Saunders, Phila Pa. (1985), pp. 34-66, 956-199. Ross D. L., Current Veterinary Therapy, V I, W.B. Saunders, Phila Pa. (1977), pp. 918-921; Eisenmenger, E. Zetner C., Veterinary Dentistry, Lea & Febiger, Phila, Pa. (1985) pp. 131-150; Harvey C. E., Textbook of Small Animal Surgery, W.B. Saunders, Philadelphia, Pa. (1985), pp. 615-620. The depth of the gingival sulcus in dogs suffering from gum disease extends from a normal condition of 1-2 mm in depth to an abnormal condition of 3-4 mm in depth, or greater. The detachment of the gingiva and the formation of periodontal pockets begin at this depth of the gingival sulcus.

Of the several signs of periodontal disease readily evident to the examining veterinarian as well as the owner, the most common presenting sign is “halitosis”, i.e., “fetid breath”. Kyle M. A., J.V.D. Vol. 5, No. 2, June 1988. This bad breath is a byproduct of the infection in the mouth. Pain due to the bacterial toxins produced accompanies this condition. If the oral pain is severe enough, irritability and improper eating habits generally develop. Eisner E. R., Veterinary Medicine, 97-104, January 1989.

Biofilm formation in pets is an extremely complex process. Almost immediately after removal of bacteria from the tooth surface by prophylaxis, a ubiquitous layer of dental pellicle is formed on tooth surfaces. The early bacterial colonizers, mostly facultative gram-positive Streptococci and Actinomyces species, adhere to the dental pellicles on the tooth surface. Following the adherence of early colonizers, the biofilm increases its cell numbers mainly by bacterial growth.

The microbial composition of biofilms gradually becomes more diversified, and after two to three weeks, the biofilm becomes a mature bacterial community. During biofilm development, various types of bacterial adhesives mediate the attachment of the bacteria to receptors in dental pellicles or on the surface of other bacteria. See Davey and O'Toole, “Microbial biofilms: from ecology to molecular genetics” Microbiol. Mol. Biol. Rev. 64:847-67 (2000). The role of biofilms in human oral care generally is applicable to pets and is detailed in the following:
Oral Health in America: A Report of the Surgeon General Executive Summary (2000) (published by the U.S. Department of Health & Human Services).
Z. Al-Yahfoufi, et al., The effect of plaque control in subjects with shallow pockets and high prevalence of periodontal pathogens, 22 J. Clin. Periodontol. 78-84 (1995).
P. Axelsson & J. Lindhe, Effect of controlled oral hygiene procedures on carries and periodontal disease in adults, 8 J. Clin. Periodontol. 239-248 (1981).
P. Axelsson & J. Lindhe, The significance of maintenance care in the treatment of periodontal disease, 8 J. Clin. Periodontol. 281-294 (1981).
D. Beighton & S. R. Brailsford, Plaque Microbiology of Root Caries, in (H. N. Newman & M. Wilson, eds.) Dental Plaque Revisited 295-312 (1999).
M. Beltrami, et al., The effect of supragingival plague control on the composition of the subgingival microflora in human periodontitis, 14 J. Clin. Periodontol. 161-164 (1987).
G. H. W. Bowden & I. R. Hamilton, Survival of Oral Bacteria, 9(1) Critical Rev. Oral Biol. Med. 54-85 (1998).
L. Braatz, et al., Antimicrobial irrigation of deep pockets to supplement non-surgical periodontal therapy, 12 J. Clin. Periodontol. 630-638 (1985).
H. J. Busscher, et al., On the relative importance of specific and non-specific approaches to oral microbial adhesion, 88 FEMS Microbiol. Revs. 199-210 (1992).
H. J. Busscher, et al., Initial microbial adhesion is a determinant for the strength of biofilm adhesion, 128 FEMS Microbiol. Lett. 229-234 (1995).
C. M. Cobb, Modern Methods for the Mechanical Control of Subgingival Plaque, in (H. N. Newman & M. Wilson, eds.) Dental Plaque Revisited 457-502 (1999).
J. W. Costerton, et al., Minireview: Biofilms, the customized microniche, 176 J. of Bacteriol. 2137-2142 (April 1994).
J. W. Costerton, et al., Microbial biofilms, 49 Annual Rev. Microbiol. 711-743 (1995).
D. Cummins, Vehicles: How to deliver the goods, 15 Periodontol 2000 84-99 (1997).
G. Dahlén, et al., The effect of supragingival plaque control on the subgingival microbiota in subjects with periodontal disease, 19 J. Clin. Periodontol. 802-809 (1992).
R. P. Darveau, et al., The microbial challenge in periodontitis, 14 Periodontol. 2000 12-32 (1997).
C. W. Douglass & C. H. Fox, Cross-sectional studies in periodontal disease: Current status and implications for dental practice, 7 Adv. Dent. Res. 25-31 (1993).
C. H. Drisko, Non-antibiotic Plaque Chemotherapy, in (H. N. Newman & M. Wilson, eds.) Dental Plaque Revisited 523-548 (1999).
S. Eick & W. Pfister, Comparison of microbial cultivation and a commercial nucleic acid based method for detection of periodontopathogenic species in subgingival plaque samples, 29 J. Clin. Periodontol. 638-644 (2002).
R. J. Gibbons, Bacterial adhesion to oral tissues: A model for infectious diseases, 68(5) J. Dent. Res. 750-760 (May 1989).
G. Greenstein, Periodontal response to mechanical non-surgical therapy: A review, 63 J. Periodontol. 118-130 (1992).
G. Greenstein & A. Polson, The role of local drug delivery in the management of periodontal diseases: A comprehensive review, 69 J. Periodontol. 507-520 (1998).
G. S. Griffiths, et al., Associations between volume and the rate of gingival crevicular fluid and clinical assessments of gingival inflammation in a population of British male adolescents, 19 J. Clin. Periodontol. 464-470 (1992).
A. D. Haffajee, et al., Plaque Microbiology in Health and Disease, in (H. N. Newman & M. Wilson, eds.) Dental Plaque Revisited 255-282 (1999).
J. M. Hardie & R. A. Whiley, Plaque Microbiology of Crown Caries, in (H. N. Newman & M. Wilson, eds.) Dental Plaque Revisited 283-294 (1999).
M. K. Hellström, et al., The effect of supragingival plaque control on the subgingival microflora in human periodontitis, 23 J. Clin. Periodontol. 934-940 (1996).
W. B. Kaldahl, et al., Long-term evaluation of periodontal therapy: II. Incidence of sites breaking down, 67 J. Periodontol. 103-108 (1996).
T. Katsanoulas, et al., The effect of supragingival plaque control on the composition of the subgingival flora in periodontal pockets, 19 J. Clin. Periodontol. 760-765 (1992).
P. Kho, et al., The effect of supragingival plaque control on the subgingival microflora, 12 J. Clin. Periodontol. 676-686 (1985).
H. M. Klimisch & G. Chandra, Use of Fourier transform infrared spectroscopy with attenuated total reflectance for in vivo quantitation of polydimethylsiloxanes on human skin, 37 J. Soc. Cosmet. Chem. 73-87 (March/April 1986).
J. W. Knowles, et al., Results of periodontal treatment related to pocket depth and attachment level. Eight years, 50(5) J. Periodontol. 225-233 (1979).
P. E. Kolenbrander & J. London, Minireview: Adhere today, here tomorrow: Oral bacterial adherence, 175 J. Bacteriol. 3247-3252 (1993).
W. F. Liljemark & C. Bloomquist, Human oral microbial ecology and dental caries and periodontal diseases, 7(2) Crit. Rev. Oral Biol. Med. 180-198 (1996).
J. Lindhe & S. Nyman, The effect of plaque control and surgical pocket elimination on the establishment and maintenance of periodontal health. A longitudinal study of periodontal therapy in cases of advanced disease, 2 J. Clin. Periodontol. 67-69 (1975).
J. Lindhe & S. Nyman, Long-term maintenance of patients treated for advanced periodontal disease, 11 J. Clin. Periodontol. 504-514 (1984).
M. A. Listgarten, Formation of Dental Plaque and Other Oral Biofilms, in (H. N. Newman & M. Wilson, eds.) Dental Plaque Revisited 187-210 (1999).
M. A. Listgarten, et al., Development of dental plaque on epoxy resin crowns in man, 46 J. Periodontol. 10-26 (1975).
H. Loe, et al., Experimental gingivitis in man, 36 J. Periodontol. 177-187 (1965).
I. Magnusson, et al., Recolonization of a subgingival microbiota following scaling in deep pockets, 11 J. Clin. Periodontol. 193-207 (1984).
P. D. Marsh, Microbial ecology of dental plaque and its significance in health and disease, 8(2) Adv. Dent. Res. 263-271 (July 1994).
P. D. Marsh, Ecological plaque hypothesis, 87 Proc. Finn. Dent. Soc. 515-525 (1991).
P. D. Marsh, Host defenses and microbial homeostasis: Role of microbial interactions, 68 (Special Issue) J. Dent. Res. 1567-1575 (1989).
P. D. Marsh, Plaque as a biofilm: Pharmacological principles of drug delivery and action in sub- and supragingival environment, 9 (Supp. 1) Oral Diseases. 16-22 (June 2003).
P. D. Marsh & D. J. Bradshaw, Physiological approaches to the control of oral biofilms, 11(1) Adv. Dent. Res. 176-185 (April 1997).
P. D. Marsh & D. J. Bradshaw, Microbial Community Aspects of Dental Plaque, in (H. N. Newman & M. Wilson, eds.) Dental Plaque Revisited 237-253 (1999)
P. D. Marsh & D. J. Bradshaw, Dental plaque as a biofilm, 15 J. Ind. Microbiol. 169-175 (1995).
R. MacAlpine, et al., Antimicrobial irrigation of deep pockets to supplement oral hygiene instruction and root debridement, 12 J. Clin. Periodontol. 568-577 (1985).
L. V. McFarland, Normal flora: Diversity and functions, 12 Microbial Ecol. in Health & Disease 193-207 (2000).
W. D. McHugh, Opening Address to the Symposium: Dental Plaque: Thirty Years On, in (H. N. Newman & M. Wilson, eds.) Dental Plaque Revisited 1-4 (1999).
H. McNabb, et al., Supragingival cleaning 3 times a week, 19 J. Clin. Periodontol. 348-356 (1992).
N. J. Mordan, et al., The Apical Plaque Border in Health and Disease, in (H. N. Newman & M. Wilson, eds.) Dental Plaque Revisited 343-374 (1999).
W. E. C. Moore & L. V. H. Moore, The bacteria of periodontal disease, 5 Periodontol. 2000 66-77 (1994).
T. Mousques, et al., Effect of scaling and root planning on the composition of the human subgingival microbial flora, 15 J. Periodontol. Res. 144-151 (1980).
R. F. Mueller, et al., Characterization of initial events in bacterial surface colonization by two pseudomonas species using image analysis, 39 Biotech. Bioeng. 1161-1170 (1992).
H. N. Newman, The organic films of enamel surface, Brit. Dent. J. 64-67 (1973).
H. N. Newman, Plaque and chronic inflammatory periodontal disease, 17 J. Clin. Periodontol. 533-541 (1990).
S. Nyman & J. Lindhe, A longitudinal study of combined periodontal and prosthetic treatment of patients with advanced periodontal disease, 50(4) J. Periodontol. 163-169 (1979).
S. Offenbacher, Periodontal diseases: Pathogenesis, 1 Ann. Periodontol. 821-879 (1996).
S. Offenbacher, et al., Periodontal infection as a possible risk factor for preterm low birth weight, 67 J. Periodontol. 1103-1113 (1996).
R. C. Page, et al., Advances in the pathogenesis of periodontitis: Summary of developments, clinical implications and future directions, 14 Periodontol. 2000 216-248 (1997).
P. N. Papapanou, et al., Current and future approaches for diagnosis of periodontal disease, NY State Dent. J. 32-37 (1999).
V. Pedrazzoli, et al., Effect of surgical and non-surgical periodontal treatment on periodontal status and subgingival microbiota, 18 J. Clin. Periodontol. 598-604 (1991).
American Academy of Periodontology's Ad Interium Committee, Periodontal therapy: A summary status report 1987-1988, 59 J. Periodontol. 306-310 (1988).
M. Quirynen, The clinical meaning of the surface roughness and the surface free energy of intra-oral hard substrata on the microbiology of the supra and subgingival plaque: Results of in vitro and in vivo experiments, 22 (Supp. 1) J. Dent., S13-S 16 (1994).
M. Quirynen & C. M. L. Bollen, The influence of surface roughness and surface free energy on supra and subgingival plaque formation in man, 22 J. Clin. Periodontol. 1-14 (1995).
S. K. Roberts, et al., Biofilm Formation and Structure: What's New, in (H. N. Newman & M. Wilson, eds.), Dental Plaque Revisited 15-35 (1999).
L. Sbordone, et al., Recolonization of the subgingival microflora after scaling and root planning in human periodontitis, 61 J. Periodontol. 579-584 (1990).
H. E. Schroeder & M. A. Listgarten, The gingival tissues: The architecture of periodontal protection, 13 Periodontol. 2000 91-120 (1997).
M. L. Sharp, et al., A test method to evaluate the efficacy of a formulation on plaque, tartar and mouth odor in dogs, Proc. World Vet. Dent. Congress 82-84 (1994).
B. G. Shearer, Biofilm and the dental office, 127 J. Am. Dent. Assoc. 181-189 (1996).
H. C. Slavkin, Biofilms, microbial ecology and Antoni Van Leerwenhock, 128 J. Am. Dent. Assoc. 492-495 (1997).
S. S. Socransky & A. D. Haffajee, The bacterial etiology of destructive periodontal disease: Current concepts, 63 J. Periodontol. 322-331 (1992).
S. S. Socransky & A. D. Haffajee, Evidence of bacterial etiology: A historical perspective, 5 Periodontol 2000 7-25 (1994).
P. Stoodley, et al., Biofilm Structure and Behavior: Influence of Hydronamics and Nutrients, in (H. N. Newman & M. Wilson, eds.), Dental Plaque Revisited 63-72 (1999).
J. M. Tanzer, On changing the cariogenic chemistry of coronal plaque, 6 (special issue) J. Dent. Res., 1576-1587 (1989).
D. van der Waaij, et al., Colonization resistance of the digestive tract in conventional and antibiotic-treated mice, 69 J. Hyg. Camb. 405-411 (1971).
J. Waerhaug, Healing of the dento-epithelial junction following subgingival plaque control, 49 J. Periodontol. 1-8 (1978).
Y. B. Wahlin & A. K. Holm, Changes in the oral microflora in patients with acute leukemia and related disorders during the period of induction therapy, 65 Oral Surg. Oral Med. Oral Pathol. 411-417 (1988).
E. Westfelt, et al., Significance of frequency of professional tooth cleaning for healing following periodontal surgery, 10 J. Clin. Periodontol. 148-156 (1983).
E. Westfelt, et al., The effect of supragingival plaque control on the progression of advanced periodontal disease, 25 J. Clin. Periodontol. 536-541 (1998).
D. E. Wilmann & E. S. Chaves, The role of dental plaque in the etiology and progress of inflammatory periodontal disease, Primary Preventative Dentistry Publ. 63-76 (1999).
M. Wilson, Bacterial biofilms and human disease, 84(3) Science Progress 235-254 (2001).
M. Wilson, Susceptibility of oral bacterial biofilms to antimicrobial agents, 44 J. Med. Microbiol. 79-87 (1996).
M. Wilson & S. Pratten, Laboratory Assessment of Antimicrobials for Plaque Related Diseases, in (H. N. Newman & M. Wilson, eds.) Dental Plaque Revisited 503-522 (1999).
Kornman, Journal of Periodontol. Res., Supplement 5:22 (1986)
Chen, Journal of the California Dental Assoc., (2001).
Kolenbrander P. E., Methods Enzymol, 253:385-97 (1995).
Kolenbrander, P. E., J. Applied Bacteriol., 78:795-865 (1993).
Grander S., J. Antimicrob. Chemother., 37:1047-50, (1996).
Davies D. G., Parsek M. R., Science, 280:295-8, (1998).
Papapanou P. N., Annu. Periodontol., 1:1-36, (1996).
Zambon J. J., Annu. Periodontol., 1:879-932, (1996).
Whittaker C. J., Klier C. M., Kolenbrander P. E., Annu. Rev. Microbiol., 50:513-52, (1996).
Wilson M., Patel H., Fletcher J., Oral Microbiol. Immunol., 11:188-192, (1996).

Periodontitis can be prevented by keeping the pet's teeth clear of plaque (biofilm) and tartar buildup, by regular cleansing of the teeth and gums or by periodic mechanical removal of tartar and/or plaque by an oral care professional. Studer E., Stapley R. D., Veterinary Medicine/Small Animal Clinician, 1124, October 1983.

According to Eisner E. R., “Basic home care consisting of regular brushing to maintain healthy teeth and gums is the cornerstone of treatment for periodontal disease in pets.” Veterinary Medicine, 698-708, July 1989. However, pet resistance to the intrusive actions of forcible brushing and owners' reluctance to perform the less-than-pleasant chore on a sufficiently regular basis, has the net effect of assuring that very few pets receive adequate oral care at home.

Unfortunately, dental hygiene in pets is something that most owners neglect. Many pet owners are unaware that just like people, pets require regular dental care. Most pet treat manufacturers have attempted at one time or another to incorporate various oral hygiene benefits in their pet care products. To date, these oral care adjuncts to pet food, chews, treats, etc., have not proven particularly successful, as the previously referenced survey of oral hygiene of pets older than four years of age brought to veterinary clinics indicates.

Pets generally require a wide range of therapeutic substances as part of their normal care, ranging from vitamins, minerals, nutraceuticals and pharmaceuticals to various systemic treatment substances. Most pets tend to resist the placement of such therapeutic substances directly in the mouth in the form of pills, capsules, gels and liquids. Additionally, these substances are usually identified and rejected by the pet when added to various dry, semi-moist and moist pet foods. The ingestible, recovery-polymer, pet chews of the invention have proven to be particularly effective as routine delivery vehicles for a wide range of therapeutic substances.

### PRIOR ART

The prior art teaches there is a need to make periodic, frequent cleansing of the teeth of dogs while making the control, disruption and removal of biofilms easier and more convenient for the pet owner, so that cleansing and biofilm control, disruption and removal are more regularly and frequently performed. To date, this need remains substantially unmet. Similarly, there is an unmet need for an effective pet chew for routinely delivering a wide range of ingestible therapeutic ingredients to pets.

A number of pet chew products have been developed over a long period of time in an attempt to address these long-felt needs. For example, U.S. Pat. No. 3,882,257 to Cagle describes a pet food product in which a slurry is dehydrated and made into a simulated bone for dogs which can help exercise the jaws and gums and help to remove tartar from the teeth. U.S. Pat. No. 4,145,447 to Fisher et al., discloses an animal food which is chew resistant and can help remove plaque or tartar from animal teeth. Still another product of this type is disclosed in U.S. Pat. No. 5,094,870 to Scaglione, et al., which discloses a process for preparing dog biscuits containing at least one inorganic phosphate salt. The dog biscuits intended to be are chewed and/or eaten by the dog with the result that tartar accumulations on its teeth are reduced or prevented. U.S. Pat. Nos. 5,296,209 and 5,407,661, both to Simone, et al., describe a product having a flexible cellular matrix in which is contained a cellulose fibrous material such as corn cob fractions having a mechanical cleansing function, which, when chewed by the pet, is intended to effect a reduction in plaque, stain and tartar on the pet's teeth. While the foregoing approaches may be meritorious, they involve creating a unique food product (as distinguished from a “chew toy”), which is a relatively complex and expensive approach, and there is no guarantee that the resultant product will be accepted and actively consumed by dogs. More importantly, these products are quickly ingested, often gulped with minimal chewing, providing little opportunity for abrasive action or very short time in the mouth for chemotherapeutic ingredients to function.

U.S. Pat. No. 5,100,651 to Boyer discloses a health product for the care of teeth of dogs, capable of being chewed or gnawed by the dogs, which contains fluoride, antimicrobial agents, and anti-decay agents.

U.S. Pat. No. 5,296,217 to Stookey discloses a method for preventing dental calculus using sequestering agents applied to commercially prepared diets of domestic animals. The sequestering agents form soluble calculus complexes in saliva and dental plaque, thereby preventing the calcifying dental plaques. Sodium hexametaphosphate has been utilized as a preferred sequestering agent. These sequestering agents can be added to dog treats, i.e., biscuits, and/or to the surface of chew toys such as rawhide.

U.S. Pat. No. 5,310,541 to Montgomery describes an animal chew product containing one or more enzymes and substrates for the purpose of generating antimicrobial compounds upon contact with animal saliva, for tartar prevention.

U.S. Pat. No. 5,431,927 to Hand, et al., describes a pet food prepared from a fiber containing nutritionally balanced mixture of carbohydrates, protein, fat, vitamins and minerals. The product has an expanded striated structure matrix which fractures when chewed by a pet, creating a mechanical tooth cleansing function which acts to reduce plaque, stains and tartar on the pet's teeth.

U.S. Pat. No. 5,467,741 to O'Rourke discloses a chew toy for dogs which is molded from soft pliable threads twisted about one or more strands of twisted synthetic fibers. The twisted fibers are impregnated with one ore more breath freshening or flavoring agents so as to dispense the agent as the dog chews.

U.S. Pat. No. 5,618,518 to Stookey discloses a chew product containing sodium hexametaphosphate, which is useful against the buildup of dental calculus.

U.S. Pat. No. 5,904,614 to Cyr et al., discloses a food dog bone made of 93% casein, poultry meal, and gelatin, and 7% of an anti-tartar composition used in the control of tartar in domestic animals such as dogs.

U.S. Pat. No. 5,908,614 to Montgomery describes a peroxidase-activating oral care composition including an enzymatic water soluble hydrogen peroxide precursor and pH adjusting agent. The composition facilitates the rapid release of hydrogen peroxide and results in the activation of a peroxidase enzyme in an oral cavity.

U.S. Pat. No. 5,944,516 to Deshaies discloses a device for cleaning the teeth of a dog, consisting of brushes, onto which toothpaste is automatically dispensed during a brushing procedure.

U.S. Pat. No. 5,989,604 to Wolf et al., discloses a pet foodstuff and treatment method for reducing the incidence of dental caries in non-human animals. Xylitol containing foodstuff is used.

Early on, pet food jerky was made by dehydrating low fat beef muscle tissue. These were highly palatable and could provide a reasonable “chew-life” if sliced and dried in thick strips. Attempts have been made to toughen reformed jerky products to improve the “chew-life”. Neilberger (U.S. Pat. No. 5,026,572) disclosed a multiple extrusion method of producing jerky by extruding a blend of wet beef and flour and then incorporating the cooked product of this first extrusion into a second extrusion step. Ray (U.S. Pat. No. 5,290,584) teaches the utilization of frozen mechanically separated meats that are comminuted to a small particle size and then mixed with pregelatinized flour prior to elevated temperature extrusion. Scaglione (U.S. Pat. No. 4,868,002) describes a process for making a tougher jerky using fibrous components of animal tissue or plant tissue such as wheat straw, alginates or industrial generated fibers.

Many long-lasting synthetic chews have been developed in attempts to address the “chew-life” issue. Axelrod (U.S. Pat. No. 4,771,733) discloses a method whereby an aqueous based flavor or odor is incorporated into a polyurethane resin based dog chew to improve the palatability of the product. Axelrod attempted further improvements to this technology (U.S. Pat. No. 5,339,771) by dispersing an animal meal within the matrix of a synthetic molded bone. Axelrod also discloses (U.S. Pat. No. 5,240,720) an injection molded chew produced from rennet casein and gelatin which can be heated by the consumer in a microwave oven to cause the chew to expand and thereby render it more easily chewable.

The effect of chewing rawhide “chips” (Chew-eez®, Superior Brands, Inc.) was compared with a leading cereal biscuit (Milk Bone®, Nabisco Brands, Inc.) on the removal of calculus in dogs reported in J. Am. Veterinary Medical Association., Vol. 197, No. 2, Jul. 15, 1990, to wit: “ . . . rawhide removed calculus considerably better than cereal biscuits for the study period.” In U.S. Pat. Nos. 5,009,973 and 5,015,485 assigned to Nabisco Brands, Inc., cereal biscuits (similar to Milk Bone®) containing pyrophosphate were reported to prevent tartar accumulation on the teeth of dogs. However, the chewing and eating of 12 such biscuits a day was required by a small dog to achieve the effect reported. This comprises 25 to 33% of the small dog's daily caloric requirement.

The act of regularly chewing an object sufficiently rigid to allow for an oral residence time of greater than thirty seconds or so has been shown to result in reduced tartar accumulation compared to a quickly consumable object, such as a biscuit (Lags, et al., J. Am. Vet. Med. Assoc., 197, pp. 213-219 (1990).

Particularly relevant additional U.S. patents include:

U.S. Pat. Nos. 6,074,662; 6,223,693; 6,277,420; 6,238,715; 6,350,438; 6,165,474; 5,047,231; 6,365,133; 6,159,508; 6,309,676; 5,635,237; 5,114,704; 5,011,679; 5,419,283 and 6,610,276. In the patented pet treat, Velvets® from Booda Bone, marketed by Aspen Products, a vinyl acetate copolymer is added to the starch to improve polymeric properties. It is noted, this additive is not digestible.

See also Levin, et al., U.S. 2003/0168020 A1, which discloses a pet chew, polymeric composition that is ductile and contains inclusions that are held in contact with a tooth while a pet's teeth penetrate the polymer during chewing. Ductility is a cumulative measurement of: tensile strength, flexured strength, shear strength, hardness and penetration (the measurement of the load needed to force a 1/25 inch diameter rod into the sample ⅛ inch). Levin, et al., does not teach or suggest recovery blend containing an emulsion nor the “recovery” property which is key to the pet chews of the present invention.

Chew toys for dogs perform several important functions. First, and most importantly, these toys facilitate several health functions, such as teeth and gum cleaning, gum massage and chewing exercise. Benefits of these functions include the prevention of periodontal disease and tartar buildup, as well as the promotion of healthy teeth and jaw development. Dogs often do not have access to natural bones and hard objects that scour their teeth when chewed and assist in healthy dental development, and owners must sometimes look to toys or snacks in order to fill this void. A variety of artificial chew toys have been created in an attempt to achieve these health benefits, with varying degrees of success. For instance, artificial chew toys have been made from rawhide, woven fibers, and ropes. However, these materials are often rapidly destroyed by the chewing action which breaks down the fibers and structure of the material, and the soft nature of these products cannot provide the same degree and variety of health benefits that can be obtained from chew toys that are comprised of harder materials.

Another important function of chew toys is to divert destructive chewing behavior and to provide amusement and entertainment for the animal. Chew toys can provide an outlet for the animal to expend its chewing energies which might otherwise by directed in a destructive manner on household objects. The degree of acceptability of the toy by the animal will determine the effectiveness and success of the product in this regard. Additionally, the toy should have an appeal to the animal and offer a means of entertainment and amusement to keep the dog happy over time, preferably over long periods of time. Therefore, it should be appreciated that there exists a need for an improved chew toy that will generate a longer period of sustained interest by dogs, thereby imparting needed health and entertainment for the animal.

Such chew products typically have a useful life (referred to hereinafter as “chew-life”) of several minutes to several hours. This “chew-life”, in addition to providing extended cleaning-type action, provides an ideal means for continually transferring biofilm disrupting ingredients contained throughout the chew to the teeth and gums of the pet as taught and claimed by the present invention.

A summary of leading commercial pet ingestible products is set forth in Table 1 below:

TABLE 1
Summary of Leading Retail Pet Ingestible Products

| Company | Brand | Positioning | Claim | Guaranteed Analysis | Ingredients |
|---|---|---|---|---|---|
| Kraft | “Original” | Milk-Bone is the | None given | Crude protein (min) 15% | Wheat flour, wheat bran, beef meal and |
| Foods | Milk-Bone ® | best way to love | (No oral care benefits | Crude fat (min) 5% | beef bone meal, beef fat preserved with |
| | Flavor | your dog | claimed) | Crude fiber (max) 3.5% | tocopherols, wheat germ, poultry by- |
| | Snacks | | | Moisture (max) 12% | product meal, lamb meal, salt, chicken |
| | | | | | meal, dried beef pulp, minerals (dicalcium |
| | | | | | phosphate), iron oxide, zinc sulfate, |
| | | | | | calcium carbonate, copper sulfate, |
| | | | | | ethylenediamine dihydroiodide (source of |
| | | | | | iodine), bacon fat (preserved with BHT, |
| | | | | | propyl-gallate, citric acid), whey (from |
| | | | | | milk), brewers dried yeast, artificial color |
| | | | | | (includes red 40, yellow 5, blue 1), malted |
| | | | | | barley flour, vitamins [choline chloride, |
| | | | | | dl-alpha tocopheryl acetate (vitamin E), |
| | | | | | vitamin A acetate, calcium pantothenate, |
| | | | | | riboflavin, vitamin B12 supplement, D- |
| | | | | | activated animal sterol (source of vitamin |
| | | | | | D3)], sodium metabisulfite (dough |
| | | | | | conditioner), casein, natural flavor, soy |
| | | | | | lecithin |
| Nabisco/ | Milk-Bone ® | Oral care bones | Help control plaque and | Crude protein (min) 18% | Rice, rice flour, sodium caseinate, water, |
| Kraft | Denta- | | tartar buildup above the | Crude fat (min) 3% | tapioca starch, propylene glycol, wheat |
| Foods | Snacks ™ | | gum line and freshen | Crude fiber (max) 4% | bran, dicalcium phosphate, corn oil, |
| | | breath | | Moisture (max) 18% | calcium carbonate, dried digest of poultry |
| | | | | Calcium (min) 1.5% | by-products, brewers dried yeast, dried |
| | | | | Calcium (max) 2% | cheese, poultry liver digest, potassium |
| | | | | | chloride, potassium sorbate, artificial color |
| S&M | Greenies ® | Multiple benefits | Dogs can't resist the taste | Crude protein not less | Processed wheat gluten, glycerin, natural |
| NuTec, | | in one nutritious | Helps clean teeth and | than 52% Crude fat not | flavor, powdered cellulose, monosodium |
| LLC | | chew | freshen breath | less than 4% Crude Fiber | phosphate, monoglycerides of edible fatty |
| | | | Abrasive action | not more than 5% | acids, magnesium and chlorophyll. |
| | | | decreases buildup of dental | Moisture not more | No artificial coloring added, no synthetic |
| | | | tartar No by-products, | than 13% | preservatives and no plastics or other inert |
| | | | preservatives, artificial | | ingredients. Product appearance and color |
| | | | flavors or colors | | may vary due to natural ingredients |
| | | | Created by a board- | | |
| | | | certified veterinary | | |
| | | | nutritionist and | | |
| | | | agricultural specialist | | |
| | | | Improves digestibility | | |
| Kal Kan | Pedigree ® | 3 in 1 Advanced | Clinically proven to: | Crude protein (min) 17% | Rice, sodium caseinate, rice flour, |
| Foods, | Dentabone ™ | Oral Care | Reduce plaque & tartar | Crude fat (min) 3% | vegetable oil, tapioca starch, |
| Inc. | | | Improve tooth and gum | Crude fiber (max) 4% | polypropylene glycol, wheat bran, calcium |
| | | | health | Moisture (max) 18% | carbonate, dicalcium phosphate, natural |
| | | | Freshen breath | | poultry flavor, potassium chloride, sodium |
| | | | | | tripolyphosphate, microcrystalline |
| | | | | | cellulose, potassium sorbate, choline |
| | | | | | chloride, BHA/BHT, eucalyptus flavor, |
| | | | | | zinc oxide, vitamin A, D3 and E |
| | | | | | supplements, zinc sulfate, ferrous sulfate, |
| | | | | | magnesium oxide, di-calcium |
| | | | | | pantothenate, salt, niacin, copper sulfate, |
| | | | | | riboflavin, pyridoxine hydrochloride |
| | | | | | (vitamin B6), thiamine mononitrate |
| | | | | | (vitamin B1), folic acid, vitamin B12 |
| | | | | | supplement |
| Denta- | DentaGreen ® | Bone treats | Help clean teeth and | Crude protein (min) 12% | Rice, rice flour, tapioca starch, casein, |
| Green ® | | (mint flavor) | freshen breath | Crude fat (min) 3% | water (sufficient for processing), |
| LLC | | | | Crude fiber (max) 4% | propylene glycol, vegetable oil, calcium |
| | | | | Moisture (max) 20% | carbonate, monocalcium phosphate, |
| | | | | | potassium chloride, sodium |
| | | | | | tripolyphosphate, sodium propionate, |
| | | | | | chlorophyll, poultry digest, potassium |
| | | | | | sorbate, peppermint oil, parsley oil, |
| | | | | | vitamin E, A and D3 |
| Heinz Pet | Meaty Bone ® | Your dog can | Dogs love the great taste of | Crude protein (min) 11% | Ground wheat, grain sorghum, animal fat |
| Products | | taste the Meaty | real meat. No other biscuit | Crude fat (min) 5% | (BHA and citric acid used as preservative), |
| | | Bone ® | gives them the mouth | Crude fiber (max) 4.5% | meat by-products, salt, cracked wheat, |
| | | | watering taste found in a | Moisture (max) 12% | beef and bone meal, caramel color, animal |
| | | | cut of beef like new | | blood plasma, beef, potassium sorbate, |
| | | | Meaty Bone. But don't take | | natural smoke flavor, red 40, minerals |
| | | | our word for it, let your | | (ferrous sulfate, zinc oxide, manganous |
| | | | dog be the judge . . . | | oxide, copper sulfate, calcium iodate, |
| | | | (no oral care benefits | | sodium selenite), vitamins (vitamin E |
| | | | claimed) | | supplement, niacin supplement, vitamin A |
| | | | | | supplement, D-calcium pantothenate, |
| | | | | | riboflavin supplement, pyridoxine |
| | | | | | hydrochloride, thiamine mononitrate, |
| | | | | | vitamin D3 supplement, folic acid, biotin, |
| | | | | | vitamin B12 supplement). AD03.04 |
| Aspen Pet | Velvets ® | New age dog | Made with corn starch | None provided | Natural corn starch, vinyl-alcohol |
| Products | from Booda ® | chew | material, derived from a | | copolymer (biodegradable), water, |
| | | | natural renewable resource. | | sorbitol, glycerin. |
| | | | Free of animal by-products. | | |
| | | | Biodegradable. Ecological. | | |
| | | | No odors or stains. | | |
| | | | (no oral care benefits | | |
| | | | claimed) | | |
| Nature's | Teeth & Breath | Dual-action | “. . . contains a special | Crude fat (min) 17% | Ground Whole Wheat, Lamb Meal, |
| Recipe | Dog Treats | formula that helps | blend of parsley seed oil | Crude protein (min) 15% | Ground Rice, Malt Syrup, Canola Oil, |
| | | to control tartar | and refreshing mint | Moisture (max) 12% | Poultry Fat (Preserved with Citric Acid, |
| | | and freshen | flavor so you can enjoy | Crude fiber (max) 5% | Mixed Tocopherols, and Rosemary |
| | | breath | being close to your dog.” | Calcium (min) 1.2% | Extract), Glycerine, Natural Flavor, Salt, |
| | | | | Phosphorous (min) 0.9% | Gum Arabic, Mint Flavor, Sodium |
| | | | | | Hexametaphosphate, Potassium Sorbate, |
| | | | | | Parsley Seed Oil, Vitamins (Vitamin E |
| | | | | | Supplement, L-Ascorbyl-2-Polyphosphate |
| | | | | | (Source of Vitamin C Activity), Inositol, |
| | | | | | Niacin Supplement, Vitamin A |
| | | | | | Supplement, d-Calcium Pantothenate, |
| | | | | | Thiamine Mononitrate, Beta-Carotene, |
| | | | | | Riboflavin Supplement, Pyridoxine |
| | | | | | Hydrochloride, Menadione Sodium |
| | | | | | Bisulfite Complex, Vitamin D3 |
| | | | | | Supplement, Folic Acid, Biotin and |
| | | | | | Vitamin B12 Supplement), Minerals (Zinc |
| | | | | | Proteinate, Ferrous Sulfate, Zinc Oxide, |
| | | | | | Iron Proteinate, Copper Sulfate, Copper |
| | | | | | Proteinate, Manganese Proteinate, |
| | | | | | Manganous Oxide, Calcium Iodate, and |
| | | | | | Sodium Selenite), Titanium Dioxide, DL |
| | | | | | Methionine, Choline Chloride. AD01.01 |
| Wysong | DentaTreat ™ | Sprinkle directly on | Helps maintain oral health | None provided | A special blend of Dental-Active Natural |
| | | foods or use as a | | | Cheeses, Trona Minerals, Calcium |
| | | tooth cleaning | | | Lactate, Potassium Citrate, Probiotic |
| | | powder for multiple | | | Cultures (Enterococcus faecium, |
| | | benefits (helps | | | Lactobacillus acidophilus, bifidus, and |
| | | prevent tooth decay | | | salivarius), Milk Calcium, Apple |
| | | and gingivitis, | | | Polyphenols, Wysong Whole Salt ™, |
| | | remineralizes teeth | | | Prebiotics (Fructooligosaccharides and |
| | | and discourages | | | Mannanoligosaccharides), and Isolated |
| | | growth of bacteria | | | Whey Proteins. |
| | | that cause plaque | | | |
| | | and bad breath) | | | |
| Dad's | Homestyle | Cleans teeth for | Offers the ultimate | Crude protein (min) 20% | Wheat flour, meat and bone meal, rice |
| Pet Care | Medium and | fresher breath | indulgence for your dog | Crude fat (min) 7.5% | bran, animal fat (preserved with BHA and |
| Company | Small Natural | | PLUS helps clean his teeth | Crude fiber (max) 3% | citric acid), calcium carbonate, salt, |
| | Biscuits | | for fresher breath | Moisture (max) 12% | potassium chloride, citric acid, choline |
| | | | | | chloride, sodium metabisulfite, ferrous |
| | | | | | sulfate, vitamin E supplement, zinc oxide, |
| | | | | | zinc proteinate, vitamin B12 supplement, |
| | | | | | vitamin A supplement, copper sulfate, |
| | | | | | copper proteinate, niacin supplement, |
| | | | | | calcium pantothenate, manganous oxide, |
| | | | | | manganese proteinate, riboflavin |
| | | | | | supplement, vitamin D3 supplement, |
| | | | | | thiamine mononitrate, pyridoxine |
| | | | | | hydrochloride, calcium iodate, sodium |
| | | | | | selenite, folic acid. |
| Old | Dog Biscuits | Perfect for any | Made with natural | Crude protein (min) 17% | Wheat Flour, Oatmeal, Chicken Fat |
| Mother | | dog, anytime | ingredients | Crude fat (min) 7.0% | (Preserved With Mixed Tocopherols), |
| Hubbard | | | (no oral care benefit | Crude fiber (max) 3% | Molasses, Charcoal, Deboned Chicken, |
| | | | claimed) | Moisture (max) 11% | Eggs, Fresh Ground Apples, Fresh Ground |
| | | | | | Carrots, Garlic, Sea Salt. |
| Hills | Science Diet ® | Helps clean teeth | Nutritionally balanced for | Protein (min) 19% | Corn meal, brewers rice, chicken by- |
| | Adult Treats | and freshen | normally active adults dogs | Fat (min) 8.5% | product meal, powdered cellulose, |
| | | breath | 1 to 6 yrs old | Crude fiber (max) 2% | chicken, chicken liver flavor, animal fat |
| | | | | Calcium (min) 0.50% | (preserved with mixed tocopherols and |
| | | | | Phosphorous (min) | citric acid), dried egg product, minerals |
| | | | | 0.35% | (potassium chloride, calcium carbonate, |
| | | | | | salt, ferrous sulfate, zinc oxide, copper |
| | | | | | sulfate, manganous oxide, calcium iodate, |
| | | | | | sodium selenite), vitamins (choline |
| | | | | | chloride, vitamin A supplement, vitamin |
| | | | | | D3 supplement, vitamin E supplement, |
| | | | | | niacin, thiamine mononitrate, calcium |
| | | | | | pantothenate, pyridoxine hydrochloride, |
| | | | | | riboflavin, folic acid, biotin, vitamin B12 |
| | | | | | supplement). |
| Hills | Science Diet ® | Helps clean teeth | Nutritionally balanced for | Protein (min) 16% | Wheat flour, soy flour, sucrose, corn |
| | Jerky Plus | and freshen | normally active adults dogs | Fat (min) 7% | gluten meal, water, propylene glycol, corn |
| | Adult Treats | breath | 1 to 6 yrs old | Crude fiber (max) 2% | meal, beef, corn flour, animal fat |
| | | | | Calcium (min) 0.40% | (preserved with BHA, propyl gallate and |
| | | | | Phosphorous (min) | citric acid), natural flavor, vegetable oil, |
| | | | | 0.35% | dried carrots, sorbic acid, beef flavor, |
| | | | | | dried parsley flakes, caramel color, |
| | | | | | carmine, canthaxanthin, phosphoric acid, |
| | | | | | sweet potato powder, titanium dioxide, |
| | | | | | minerals (calcium chloride, potassium |
| | | | | | chloride, dicalcium phosphate, salt, ferrous |
| | | | | | sulfate, zinc oxide, copper sulfate, |
| | | | | | manganous oxide, calcium iodate, sodium |
| | | | | | selenite), ethoxyquin (a preservative), |
| | | | | | vitamins (choline chloride, vitamin A |
| | | | | | supplement, vitamin D3 supplement, |
| | | | | | vitamin E supplement, niacin, thiamine, |
| | | | | | calcium pantothenate, pyridoxine |
| | | | | | hydrochloride, riboflavin, folic acid, |
| | | | | | biotin, vitamin B12 supplement). |
| Purina ® | Essentials ™ | Tailored to meet | Help maintain strong teeth | Crude protein (min) 14% | Wheat flour, beef tallow preserved with |
| | Healthy Teeth | certain health | and healthy bones | Crude fat (min) 3% | mixed-tocopherols (source of Vitamin E), |
| | & Bones | needs in a tasty | | Crude fiber (max) 2% | beef and bone meal, ground wheat, |
| | | treat they'll love. | | Moisture (max) 12% | lecithin, defluorinated phosphate, dried |
| | | It's the healthy | | Calcium (min) 1.0% | cheese powder, dicalcium phosphate, salt, |
| | | way to treat your | | Phosphorous (min) 0.9% | L-Lysine monohydrochloride, sodium |
| | | dog right. | | Vitamin D3 (min) | metabisulfite, Vitamin D-3. |
| | | | | 300 IU/kg | D-4822 |
| DermaPet | Dentees ™ | Dental Treat | Freshen breath; decreases | Crude protein (min) 52% | Processed wheat gluten, glycerin, natural |
| | DentAcetic | with Odofactants | buildup of plaque; blocks | Crude fat (min) 4% | flavor, powdered cellulose, monosodium |
| | Dog Chews | | formation of tartar | Crude Fiber (min) 5% | phosphate, monoglycerides of edible fatty |
| | | | | Moisture 7% | acids, magnesium stearate, sodium |
| | | | | | hexametaphosphate, acetic acid, grapefruit |
| | | | | | seed extract, clove |

It is interesting to note that of the fourteen (14) branded retail pet treats detailed in Table 1: (a) only four made no oral care benefit claims; (b) only three of the ten making oral care claims contained cleaners, anti-tartar or anti plaque ingredients; and (c) only three contained small amounts of “toothpaste abrasives.”

### OBJECTS OF THE INVENTION

An object of the present invention is to provide ingestible, recovery pet chews that resist fracturing and crumbling over their chew-life.

A further object of the present invention is to provide extruded and densified, ingestible, recovery pet chews that resist fracturing and crumbling throughout their chew-life.

Another object of the present invention is to provide extruded and densified, ingestible, recovery pet chews with acceptable: recovery and chew-life properties.

An object of a preferred embodiment of the present invention is to provide extruded and densified, ingestible, recovery pet chews suitable for delivering therapeutic substances and/or controlling, disrupting and removing biofilms from tooth surfaces of pets, while indicating acceptable recovery properties and resisting fracturing and crumbling throughout their chew-life.

A further object of a preferred embodiment of the invention is to provide extruded and densified, ingestible, recovery pet chews suitable for releasing over their chew-life into the pet's oral cavity, ingredients that help simultaneously: control and disrupt biofilms and/or therapeutic ingredients.

Still another object of the invention is to provide innovative methods for extruding and densifying ingestible, recovery pet chews.

Yet another object of the present invention is to enhance: the penetration value, recovery property, Shore-D Hardness and chew-life of recovery pet chews.

Still another object of a preferred embodiment of the invention is to provide a pet “self-treatment” for biofilm buildup comprising routinely providing the pet ingestible, recovery, oral care chews suitable for controlling, disrupting and removing biofilms attached to tooth surfaces.

Still a further object of the invention is to provide a means for plasticizing recovery pet chews using a digestible plasticizing substance.

Another object of a preferred embodiment of the invention is to provide recovery pet chews with biofilm disrupting and controlling emulsions.

Still another object of a preferred embodiment of the invention is to provide recovery pet chews containing oral care ingredients and/or therapeutic substances with extended chew-life, while maintaining digestibility and minimizing tendency for blockage of pet digestive systems.

Yet another object of the invention is to provide a method for controlling, disrupting and removing biofilms.

A further object of the invention is to provide therapeutic, ingestible, recovery pet chews with extended chew-life, while maintaining and minimizing tendency for blockage of pet digestive systems.

A further object of the invention is to provide an ingestible, recovery pet chew vehicle suitable for routinely delivering a wide range of therapeutic substances to pets.

### BRIEF DESCRIPTION OF DRAWINGS

FIG. 1 is a photograph of a mechanical jaw-like device used to establish penetration values of recovery pet chews.

FIG. 2 is a photograph of the mechanical jaw device penetrating a recovery pet chew of the invention.

FIG. 3 is a plan view of a recovery pet chew immediately after penetration.

FIG. 4 is a plan view of a recovery pet chew 5 minutes after penetration, illustrating the penetration properties of the pet chew.

FIGS. 5 through 11 show the device used to ascertain recovery properties of the pet chews of the invention with FIGS. 7 through 11 showing recovery chews at various stages of recovery.

FIG. 5 shows a side view of the recovery probe mounted on the recovery press means.

FIG. 6 is an enlarged side view of the recovery probe mounted on the recovery press.

FIG. 7 is a side view of the recovery probe touching a recovery chew of the invention prior to penetration.

FIG. 8 is a side view of the recovery probe penetrating the recovery chew with the probe fully extended into the chew.

FIG. 9 is a plan view of a recovery chew of the invention immediately after the recovery probe has been removed with the hole formed by the probe readily apparent.

FIG. 10 is an exploded plan view of the hole formed in the recovery chew by the recovery probe, immediately after the probe has been reviewed.

FIG. 11 is an exploded plan view of the hole formed in the chew at two minutes after the probe has been removed, with the hole starting to recover, close and become more shallow.

### SUMMARY OF THE INVENTION

The present invention, which is distinct from and neither implied nor suggested in the prior art nor in the commercial products described in Table 1 above, is directed to extruded and densified, ingestible, recovery pet chews comprising: an emulsion, starch, protein, water and a plasticizing substance.

In one preferred oral care embodiment of the invention, the recovery pet chews also indicate biofilm disrupting, controlling and removing properties. The recovery pet chews of this preferred embodiment of the invention are ideally suited for: controlling, disrupting and removing biofilms from pet tooth surfaces, while resisting fracturing and crumbling over their chew-life. As these oral care, recovery pet chews are consumed, they physically remove biofilms while simultaneously over their chew-life, releasing substantive biofilm disrupting and biofilm and tartar controlling ingredients throughout the oral cavity of the pet.

In another preferred embodiment of the invention, the recovery pet chews of the invention also contain a therapeutic substance which is released into the pet's oral cavity over the chew-life of the pet chew. The recovery pet chews provide an excellent vehicle for delivering ingestible, therapeutic substances to the pet's oral cavity.

The various recovery pet chews of the invention, including oral care and therapeutic, recovery pet chews, indicate exceptional: recovery, penetration, Shore-D Hardness and chew-life properties.

The recovery feature of the various pet chews of the present invention extends the chewing time, while discouraging “chunk biting” and swallowing, thereby dramatically extending the chew-life and the various treatments associated with the wide range of ingredients included in the recovery pet chews. Tooth penetration and removal from the recovery pet chews is comparable to the tooth-cleaning-action the pet normally encounters when ripping through oxtails or other sinewy substances.

### Description of Key Terms

For the purposes of the present invention, the following key terms are defined as set out below:

“Periodontal disease” (“gum disease”) is a broad term used to describe those biofilm-based diseases which attack the gingiva and the underlying alveolar bone supporting the pet's teeth. The disease exists in a number of species of warm blooded animals such as canines and felines, and includes a series of diseases exhibiting various syndromes which vary from each other according to the stage or situation of the disease or the age of the pet. The term is used for any inflammatory disease which initially occurs at a marginal gingiva area and may affect the alveolar bone. Periodontal disease affects the periodontium, which is the investing and supporting tissue surrounding a tooth (i.e., the periodontal ligament, the gingiva, and the alveolar bone). Two common periodontal diseases are gingivitis (inflammation of the gingiva) and periodontitis (inflammation of the periodontal ligament manifested by progressive resorption of alveolar bone, increasing mobility of the teeth, and loss of the teeth at advanced stage). Other terms used for various aspects of periodontal disease are “acute necrotizing ulcerative gingivitis” and “alveolar pyorrhea”. Periodontal disease may involve one or more of the following conditions: inflammation of the gingiva, formation of periodontal pockets, bleeding and/or pus discharge from the periodontal pockets, resorption of alveolar bone, loose teeth and loss of teeth. Periodontal disease is generally considered to be caused by/associated with bacteria which are generally present in biofilms (dental plaque) which forms on the surface of the teeth and in the periodontal pocket. Thus, known methods for treating periodontal disease often include the use of antimicrobials and/or anti-inflammatory drugs.

“Biofilm (plaque),” the precursor of dental calculus/tartar and the source of gum disease, is defined as a community of bacteria embedded in exopolysaccharide that adheres to tooth surfaces and are a major source of the infections associated with gum disease in pets. The early bacterial colonizers of biofilm, which are mostly faculative gram-positive Streptococci and Actinomyces species, adhere to the dental pellicles on the tooth surface. Following the adherence of early colonizers, the biofilm increases its cell numbers mainly by bacterial growth.

“Dental calculus,” or tartar as it is sometimes called, is defined as a deposit of hardened plaque (biofilm) which forms on the surfaces of the teeth at the gingival margin. Supragingival calculus appears principally in the areas near the orifices of the salivary ducts; e.g., on the lingual surfaces of the lower anterior teeth and on the buccal surfaces of the upper first and second molars, and on the distal surfaces of the posterior molars. Mature calculus consists of an inorganic portion which is largely calcium phosphate arranged in a hydroxyapatite crystal lattice structure similar to bone, enamel and dentin. An organic portion (biofilm) is also present and consists of desquamated epithelial cells, leukocytes, salivary sediment, food debris and various types of microorganisms. As the mature calculus develops, it becomes visibly white or yellowish in color unless stained or discolored by some extraneous agency. In addition to being unsightly and undesirable from an aesthetic standpoint, the mature calculus deposits are constant sources of irritation of the gingiva and thereby are a contributing factor to gingivitis and other diseases of the supporting structures of the teeth, the irritation decreasing the resistance of tissues to endogenous and exogenous organisms.

“Surfactants” are defined as surface active agents suitable for ingestion. Said Surfactants have the property of being water soluble with a propensity to emulsify water-insoluble coating agents (as defined below), and to hold the coating agent in an aqueous suspension as an emulsion when the mixture is dispersed in water or saliva. Suitable surfactants, illustrative of the types of substances suitable for use in ingestible, recovery-polymeric, pet oral care chews of the present invention, are further detailed below.

“Coating Agents” are defined as water insoluble or very slightly soluble substances which, when presented to the oral cavity in an emulsified state, will coat the teeth, gums and oral cavity tissue with a thin film of the coating agent. This film has several beneficial properties which are functionally described below.

“MICRODENT®” and “ULTRAMULSION®” are defined as hot melt emulsions of biofilm disrupting coating substances such as polydimethylsiloxane in surfactants such as nonionic poloxamer surfactants and include those emulsions described in U.S. Pat. Nos. 4,950,479; 5,032,387; 5,057,309; 5,538,667; 5,651,959 and 5,711,936. These patents are incorporated herein by reference. The clinical plaque effect obtained when certain of these combinations of surfactants and coating substances are introduced into the mouths of humans are detailed in Food & Drug Administration (FDA) Docket No. 81N-0033, OTC Volumes 210246 to 210262 and 210339 dated Jun. 17, 1991, filed in response to the FDA call-for-data as reported in the Federal Register, Sep. 19, 1990, 55 Fed. Reg., 38560, Vol. VI of said filing; the summary is specifically incorporated herein by reference.

“Oral care emulsion” is defined as a physical mixture of two or more phases which is used to satisfy cleansing and breath freshening requirement. See also MICRODENT® and ULTRAMULSION®.

“Chew-life” is defined as the duration that a round pet chew (about 2.5 inches in diameter and about 0.5 inches thick) can be chewed, gnawed, licked, etc., by a pet (weighing less than about 10 pounds) before it is consumed. Chew-life defines the transfer period for various therapeutic ingredients, such as biofilm disrupting emulsions, which are contained in MICRODENT® containing, recovery, oral care pet chews of the invention and released into the pet's oral cavity during chewing.

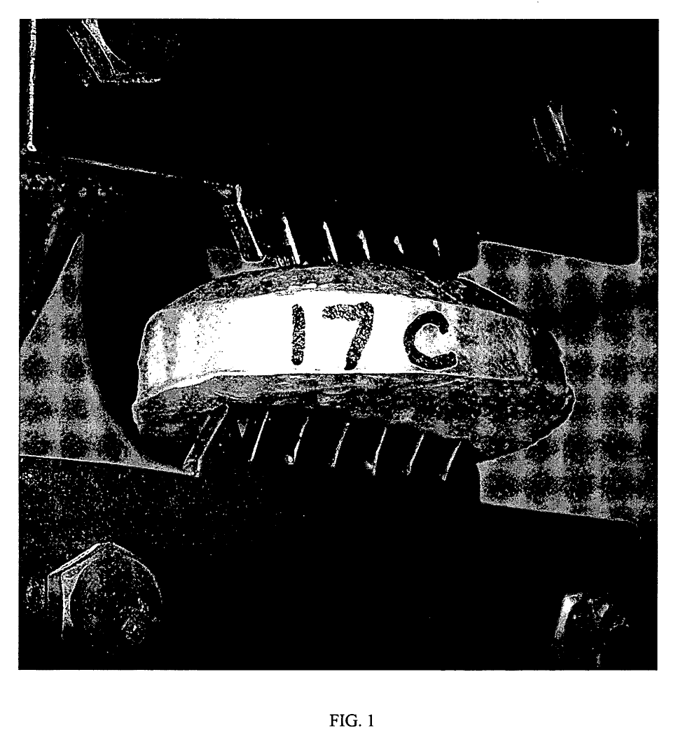

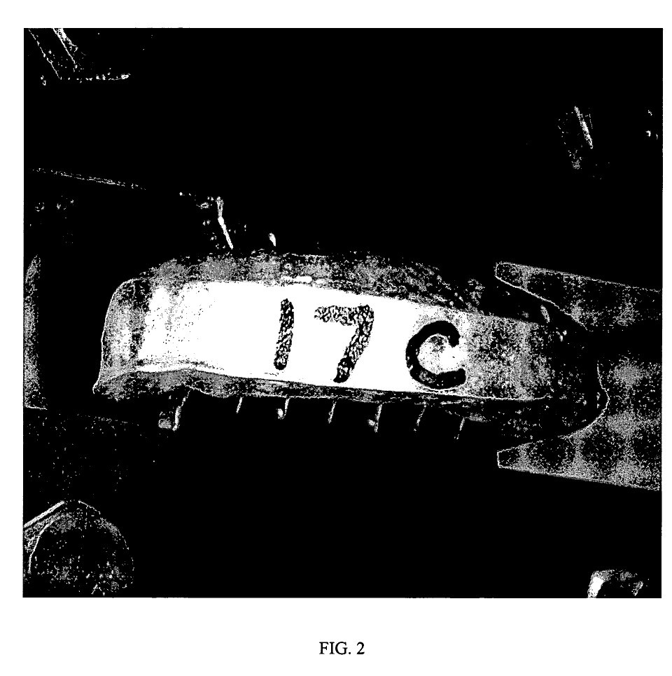

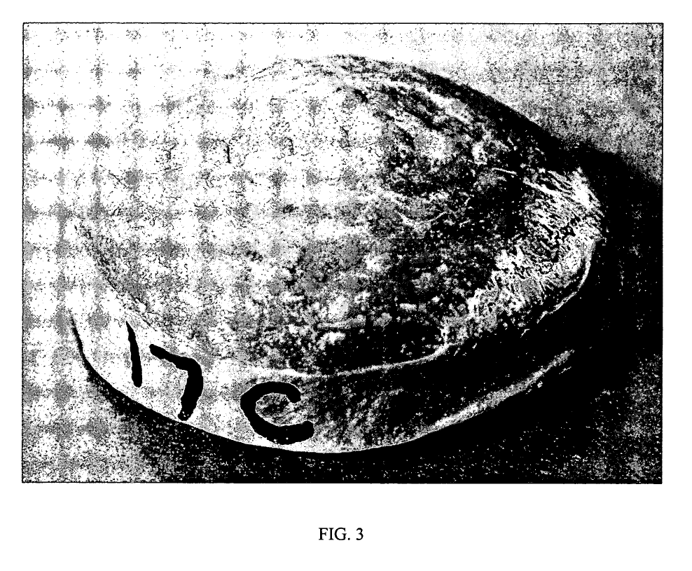

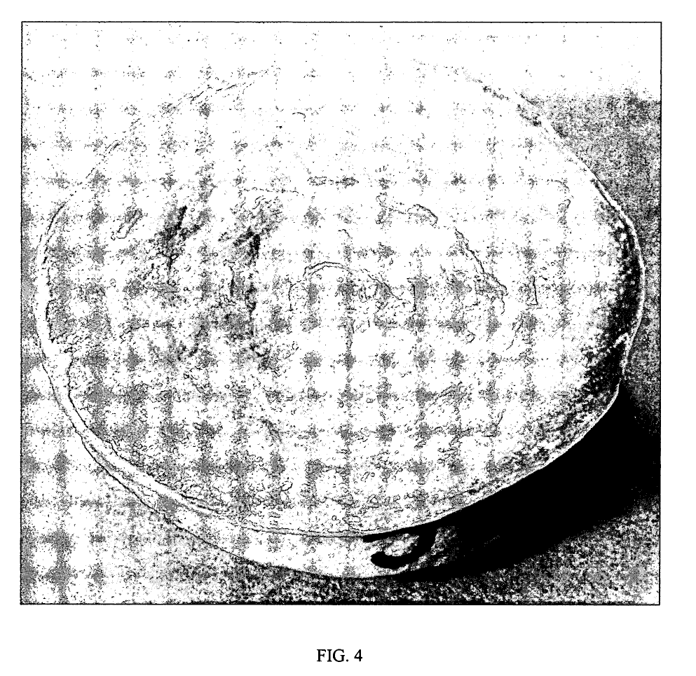

“Physical penetration (in psi)” is defined as the amount of pressure applied to a sharp, saw-toothed, “jaw-type” device shown in FIG. 1 to penetrate a recovery chew to a depth of 25 percent of the chew thickness as shown in FIGS. 2 through 4.

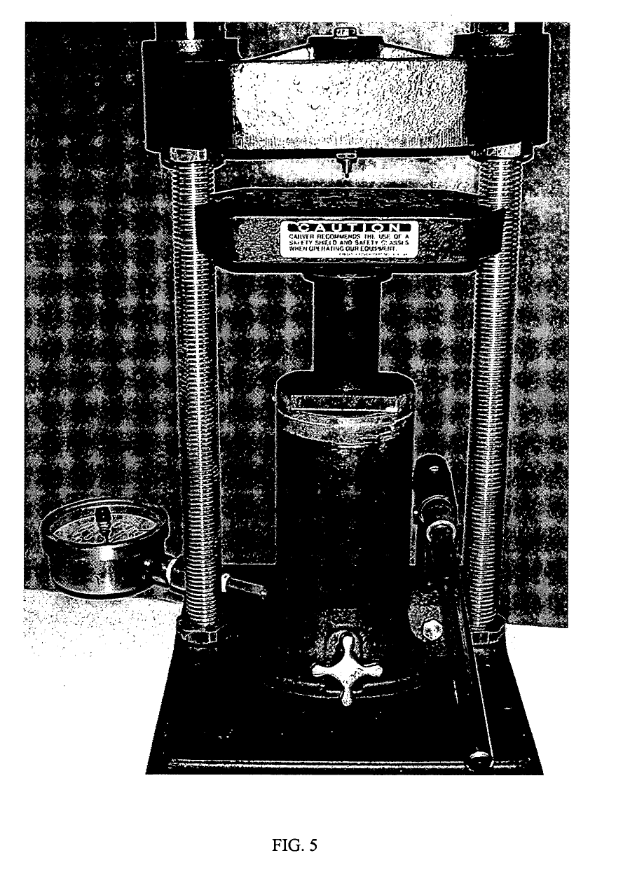

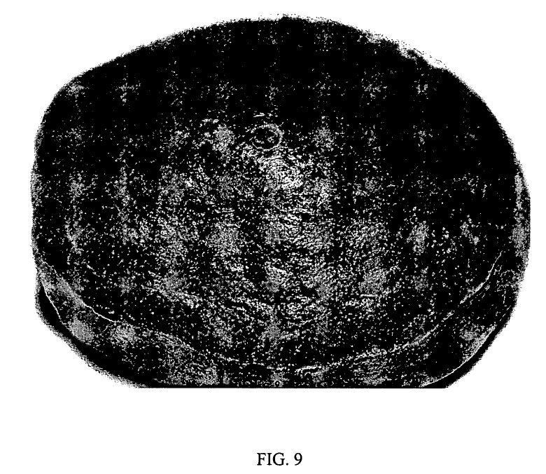

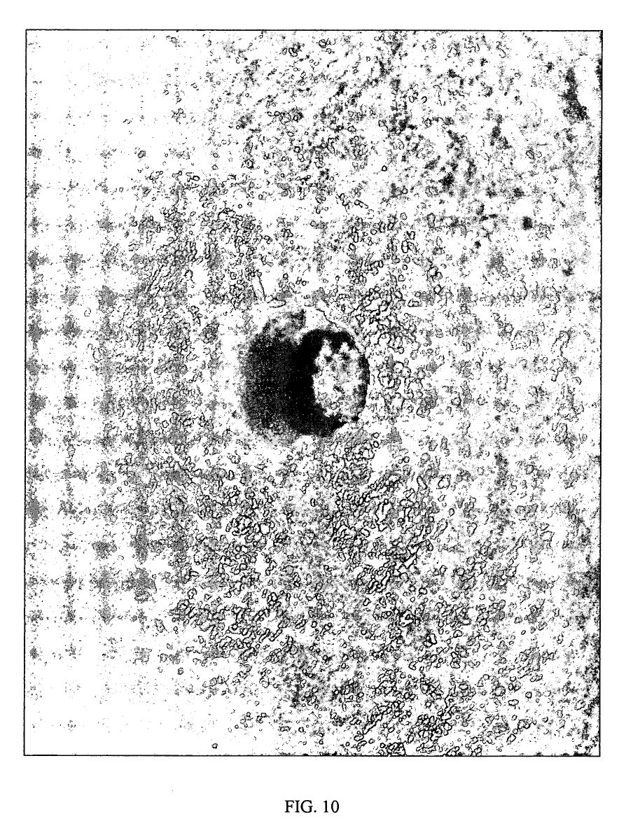

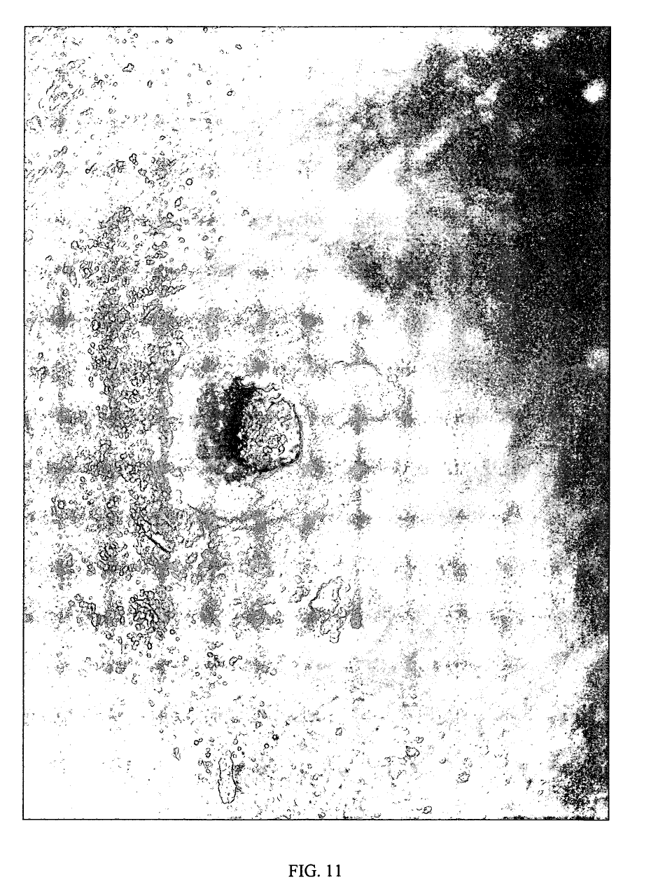

“Recovery blend” is defined as an extruded and densified composition comprising an emulsion, starch, protein, a plasticizing substance and water, which exhibits the ability, upon penetration as shown in FIGS. 5 through 11 to reclose the resultant hole upon removal of said object two minutes after penetration.

“Recovery Value” is defined in terms of the percent of the volume of a hole formed in a recovery chew of the invention (when penetrated using a standard probe 0.150 inches in length) that recovers or closes within two minutes of penetration. See FIGS. 5 through 11 of the drawings and Table 2 below.

“Extruded” is defined as the formation of an object by forcing hot material through an aperture.

“Densified” is defined as a process for removing voids and gaseous materials from extruded substances. See detailed description in Example 1.

“Ingestible” is defined as ingredients that are normally consumed by warm-blooded animals to provide energy.

“Digestible” is defined as a substance which is digestible if, when exposed to gastric pH and enzymes, it breaks down into smaller units that are able to be used for energy production.

“Starch” is defined as a biopolymer composed of repeating units of D-glucose.

“Available water” is defined as the water determined by instrumental methods to be loosely bound to a substrate material.

“Protein” is defined as a polymer of naturally occurring amino acids that is essential for all living things.

“Density” is defined as the weight per unit volume.

“Shore-D Hardness” is defined as a method of measuring relative hardness of rubber, plastics and other softer materials utilizing an indenter, a calibrated spring and a depth indicator.

“Plasticizing substance” is defined as a substance which, when added to a normally non-plastic, polymeric substance like a starch or protein, alters its normally non-plastic properties and behavior to those more typical of a synthetic thermoplastic. That is, the non-plastic, polymeric substance is subsequently able to be shaped and formed by a combination of elevated temperature and pressure, e.g., by extrusion and densification, into the pet chews of the present invention and retain their thermoplastic properties without returning to their original, non-plastic characteristics.

“Emulsion” is defined as a colloidal dispersion of two or more immiscible liquids.

“Oral care abrasive” is defined as an abrasive possessing a hardness and particle size that is safe and effective for oral hygiene use.

“Therapeutic” is defined as a property of a substance or process which has a curative effect.

### DETAILED DESCRIPTION OF THE PREFERRED EMBODIMENTS

The extraordinary saliva flow in carnivores is substantially greater than in humans and, as a result, most therapeutic substances released from pet oral care treats and/or chews during chewing or introduced via toothpaste, rinses, etc., lack effective residence time in the mouth, i.e., they are readily flushed by this extraordinary flow of saliva out of the oral cavity, usually before the therapeutic substance can have any substantial therapeutic effect in the pet's mouth.

FIGS. 1 through 4 are photographs of a penetration testing device used to establish penetration values of the recovery pet chews of the invention. Physical penetration values are established by applying pressure to the sharp, saw-toothed, testing device shown in FIG. 1. The amount of force measured in pounds per square inch (psi) required to penetrate a depth equal to about 25% of the chew thickness are shown in FIGS. 2 through 4. Various recovery chews of the invention have been evaluated for penetration values over a period of time. These are reported in Table 3 below.

FIGS. 5 through 11 are photographs of a recovery measuring device and of recovery pet chews of the invention being evaluated for their recovery value.

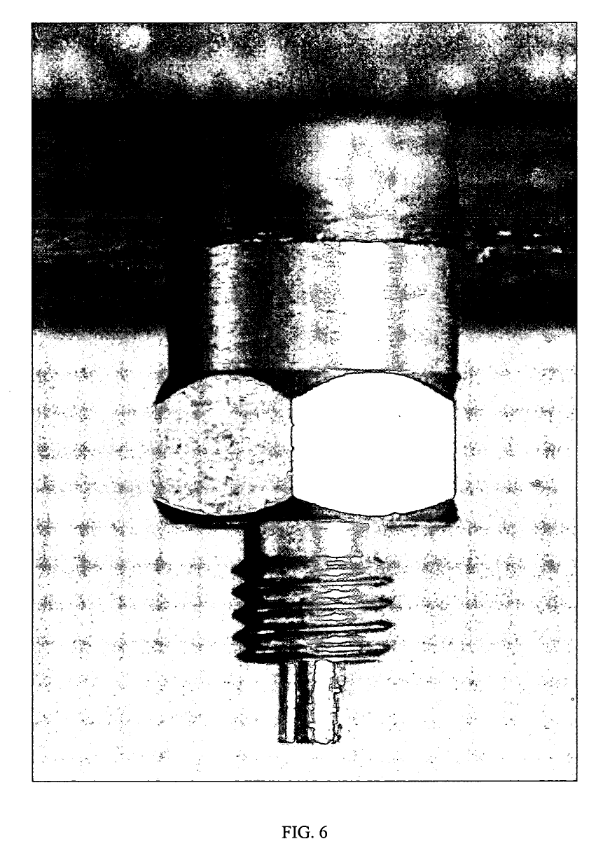

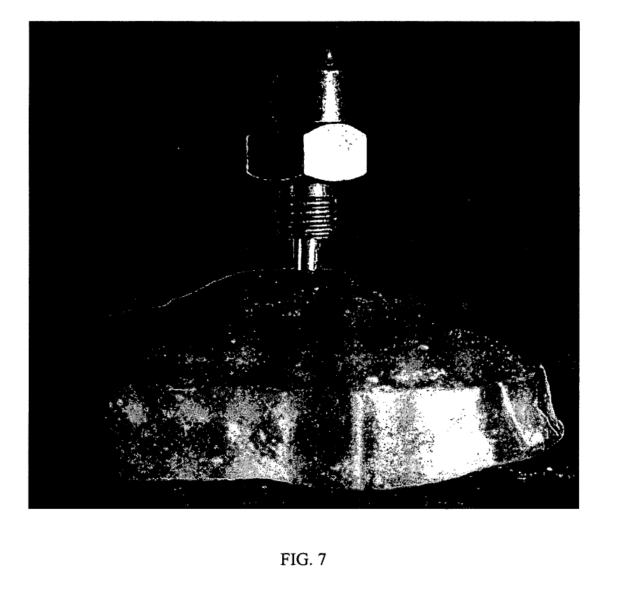

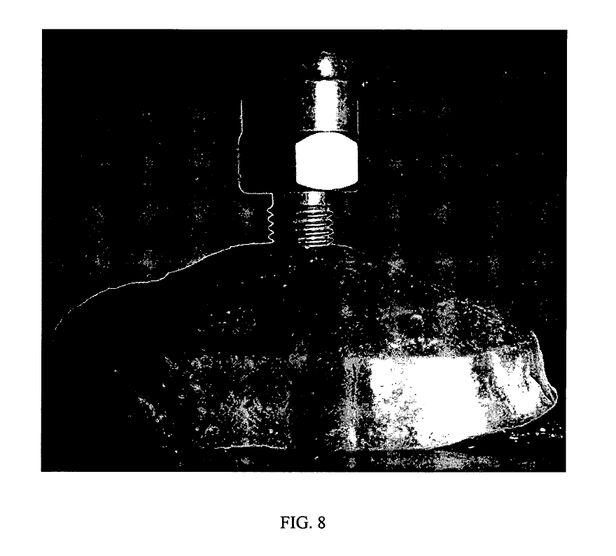

The recovery probe shown in FIG. 6 penetrates the recovery chew of the invention to a depth of 0.150 inches as shown in FIGS. 7 through 10. The hole formed upon probing is shown in FIGS. 8 through 10. The percent of the hole that has recovered from probing after two minutes is established.

These recovery values for two production lots of recovery pet chews of varying ages, i.e., fresh and 1 year old, is detailed in Table 2 below.

The samples from two lots (1 year old and fresh) to be evaluated for recovery are designated A through J, respectively. The percent recovery 2 minutes after penetration is recorded. All samples, irrespective of age, indicated substantial recovery, i.e., well over 50%, with approximately 5% less recovery recorded for the 1 year old samples, when compared to fresh. See Table 2 below.

TABLE 2

| | |
|---|---|
| Lot 1 | |
| (1 year old) | Fresh |

| | |
|---|---|
| Sample | % of hole that has recovered and |
| Designation | resealed after 2 minutes |

| | | |
|---|---|---|
| A | 55 | 59 |
| B | 63 | 59 |
| C | 67 | 63 |
| D | 62 | 63 |
| E | 54 | 61 |
| F | 57 | 62 |
| G | 55 | 66 |
| H | 61 | 61 |
| I | 55 | 57 |
| J | 56 | 62 |
| Avg. % Rec. = | 58.5 | 61.3 |

The substantivity of the oral care emulsions present in the recovery pet oral care chews of the present invention to tooth and gum surfaces plays a most critical role in effective biofilm therapy attributed to the chews of the invention. The preferred “pet applied” oral care “emulsions” comprise—MICRODENT® or ULTRAMULSION® which are extruded and densified substantially throughout the recovery pet oral care chews of the invention. Examples of various pet chews of the invention containing these emulsions and other ingredients comprising the recovery-blend are detailed in Examples 1-31 and in Tables 3 through 6 below. These emulsions are introduced into the oral cavity during chewing and tend to form ablative coatings on tooth surfaces which disrupt and control biofilm formation by lowering the surface energy of the pet's teeth. Faced with these reduced surface energy tooth surfaces, the microflora responsible for forming biofilms have a difficult time sticking and biofilm formation is disrupted and controlled.

Simultaneously, the recovery pet chews with: (a) physical penetration values between about 600 and about 1000 psi, (b) Shore-D Hardness values of at least about 25, (c) recovery values of at least about 30%, and (d) a chew-life of at least about four minutes, effectively help physically remove biofilms with an abrasive-type rubbing action, during chewing. Other therapeutic ingredients present in the recovery chew are released into the pet's oral cavity for consumption over the chew-life of the chew.

Ingestible, recovery pet chews of the invention provide:
(1) A means for introducing various ingestible, therapeutic ingredients into the pet's oral cavity, including: pharmaceuticals, minerals, vitamins, nutraceuticals, systemic treatment substances and the like over the chew life of the product;
(2) A convenient and effective means for introducing various therapeutic substances that help control and disrupt biofilms and/or tartar, such as tartar complexing substances, etc.;
(3) A means for introducing biofilm controlling and disrupting emulsions into the oral cavity. These biofilm controlling and disrupting emulsions are distributed throughout the ingestible, recovery chew. See Examples 1 through 20 in Table 3 below. Intimate contact between these emulsions and the pet's saliva, gums and teeth occurs throughout the chew-life of the chew, assuring that these biofilm disrupting and controlling emulsions are coating the oral cavity before they are eventually washed away and consumed; and
(4) A convenient and effective means for achieving mechanical abrasion of teeth for extended periods which is particularly effective in controlling biofilm and tartar/calculus buildup. The act of regularly chewing pet treats sufficiently rigid to allow for an oral residence time greater than 30 seconds has been shown to result in reduced tartar accumulation, compared to a quickly consumable object such as a biscuit. See Lags, et al. J. Am. Veterinary Med. Assoc. 197, pp. 215-219 (1990).

This combination of releasing, throughout the chew-life of the recovery-polymeric chew, therapeutic ingredients, including oral care ingredients such as biofilm controlling and disrupting emulsions, along with the physical, abrasive removal of biofilms through chewing the recovery pet chew distinguishes the pet chews of the present invention from the prior art as well as all of the ingestible, commercial products described in Table 1 above.

Recovery pet chews of the invention containing therapeutic ingredients are described in illustrative Examples 22-31 in Table 6.

Recovery pet oral care chews of the invention are described in Examples 1 through 20, detailed in Table 3, and Example 21, detailed in Table 4. All these recovery pet chews are preferably manufactured using an extruder/product densification operation such as described and shown in U.S. Pat. No. 5,622,744. Such equipment is available commercially from Extru-Tech, Inc., Sabetha, Kans. See also U.S. Pat. No. 4,971,711. The extrudate from the densification stage can be processed into suitable pellets and subsequently utilized by processing in a high pressure compression injection molding operation.

Various shapes for recovery pet chews, including therapeutic, chemotherapeutic, oral care chews other than the “hockey-puck” shown in the penetration photographs (FIGS. 1 through 4), can be processed using the extruder-densifier processing operation described in Example 21.

For example, two compressing rollers positioned to engage the extrudate from the round die used to manufacture the “hockey-puck” shape will produce a continuous “ribbon” which can be sliced or die cut into various flat shapes, preferably with a compressed “relief” decoration or brand name.

The various recovery pet chew blends, described in Examples 1 through 20 in Table 3; Example 21 in Table 3; Examples 22 through 28 in Table 5; and Examples 29 through 31 in Table 6, are initially preconditioned with water or steam, which is added prior to extrusion to form an intermediate product which is transferred to a product densification unit, i.e., a high-speed, low-shear extruder adapted to densify and shape recovery products. The shaft speed of the high speed screw in the product densification unit helps to control the final density of the recovery pet chew discs produced.

### EXAMPLES 1 THROUGH 20

Table 3 below, with Examples 1 through 20, describes a wide range of recovery blend formulations suitable for the recovery pet chews of the present invention. The MICRODENT® described in some of the Examples comprises an emulsion of polydimethylsiloxane (1,000 cs) in a nonionic surfactant poloxamer, where the wt. ratio of the silicone to surfactant is between 1:5 and 1:15. The ULTRAMULSION® described in some of the Examples comprises polydimethylsiloxane (2.5 million cs) in a nonionic surfactant poloxamer, where the wt. ratio of silicone to surfactant is between about 1:3 and about 1:20.

The method used to manufacture recovery pet chews of the invention with these formulations is detailed in Example 21.

TABLE 3
Examples 1-20 - Recovery, Oral Care, Pet Chew Formulations

| | | | | | Weight | Oral Care | | |
|---|---|---|---|---|---|---|---|---|
| | | | Weight | | Ratio of Plasticizing | Emulsions & other | | |
| | | | Ratio of | Plasticizing | Agent to | Oral Care | Other | Added |
| | Starch(es) | Protein(s) | Starch(es) | Agent | Starch/Protein | Ingredients(s) | Additive(s) | Water |
| Ex. No. | (% by wt.) | (% by wt.) | to Protein(s) | (% by wt.) | Mixture | (% by wt.) | (% by wt.) | (% by wt.) |
| 1 | Durham flour | wheat gluten | 10.8 | Fructose | 0.64 | MICRODENT ® (2) | natural chicken | 4.3 |
| | (29) | (5) | | (30.6) | | DCP | (0.1) | |
| | tapioca starch | | | | | (2) | | |
| | (25) | | | | | TSPP | | |
| | | | | | | (2) | | |
| 2 | Durham flour | wheat gluten | 10 | Fructose | 0.54 | MICRODENT ® | natural chicken | 4.0 |
| | (27) | (5) | | (28.9) | | (4) | (0.1) | |
| | tapioca starch | | | | | DCP | | |
| | (23) | | | | | (4) | | |
| | | | | | | TSPP | | |
| | | | | | | (4) | | |
| 3 | corn starch | — | — | cane syrup (4) | 0.54 | ULTRAMULSION ® | natural chicken | 5.0 |
| | (29) | | | honey (4) | | (4) | (0.1) | |
| | rice flour | | | grace juice | | DCP | | |
| | (10) | | | concentrate (8) | | (4) | | |
| | tapioca starch | | | corn syrup (8.9) | | TSPP | | |
| | (15) | | | molasses (4) | | (4) | | |
| 4 | corn starch | wheat gluten | 0.026 | Sorbitol-70 | 0.15 | ULTRAMULSION ® | dried chicken | 4.0 |
| | (2) | (75.75) | | (12) | | (2) | (1.5) | |
| | | | | | | | Canola oil | |
| | | | | | | | (2.75) | |
| 5 | corn starch | wheat gluten | 0.58 | Sorbitol-70 | 0.16 | ULTRAMULSION ® | dried chicken | 4.5 |
| | (29.1) | (49.9) | | (13) | | (2) | (1.5) | |
| 6 | corn starch | wheat gluten | 1.00 | Sorbitol-70 | 0.84 | ULTRAMULSION ® | dried chicken | (4.5) |
| | (25) | (25) | | (42) | | (2) | (1.5) | |
| 7 | corn starch | Rice Flour | 0.70 | Sorbitol-70 | 0.48 | ULTRAMULSION ® | dried chicken | (4.5) |
| | (25.6) | (36.4) | | (30) | | (2) | (1.5) | |
| 8 | corn starch | Pre-conditioned | 0.21 | Sorbitol-70 | 0.29 | ULTRAMULSION ® | dried chicken | (3.0) |
| | (12.75) | (A) | | (20.75) | | (2) | (1.5) | |
| | | wheat gluten | | | | | | |
| | | (60) | | | | | | |
| 9 | corn starch | wheat gluten | 0.74 | Sorbitol-70 | 0.14 | ULTRAMULSION ® | dried chicken | (4.0) |
| | (0) | (48) | | (12.5) | | (0) | (0) | |
| | Parsley Flour | | | | | | | |
| | (35.5) | | | | | | | |
| 10 | corn starch | Pre-conditioned | 0.75 | Sorbitol-70 | 0.15 | ULTRAMULSION ® | dried chicken | (4.0) |
| | (5) | (A) | | (12.5) | | (0) | (0) | |
| | Rice Flour | wheat gluten | | | | | | |
| | (30.5) | (48) | | | | | | |
| 11 | Wheat bran | Pre-conditioned | NA | Sorbitol-70 | 0.22 | ULTRAMULSION ® | dried chicken | (4.0) |
| | (3) | (B) | | (16) | | (2) | (1.5) | |
| | | wheat gluten | | | | | Canola oil | |
| | | (71.5) | | | | | (2) | |
| 12 | Wheat bran | wheat gluten | 0.10 | Glycerin | 0.13 | ULTRAMULSION ® | dried chicken | (4.75) |
| | (7) | Plasticized | | (10) | | (2) | (3.5) | |
| | Xanthan Gum | (71.25) | | | | | | |
| | (1.5) | | | | | | | |
| 13 | Parsley Flour | wheat gluten | 0.14 | Sorbitol-Power | 0.35 | ULTRAMULSION ® | dried chicken | (4.0) |
| | (4) | (58.5) | | (23) | | (2) | (1.5) | |
| | corn starch | | | | | | | |
| | (4) | | | | | | | |
| | Wheat bran | | | | | | | |
| | (3) | | | | | | | |
| 14 | Bread flour | wheat gluten | 1.27 | Sorbitol-70 | 0.29 | ULTRAMULSION ® | dried chicken | (5.0) |
| | (41.8) | (33) | | (16.7) | | (2) | (1.5) | |
| 15 | Bread flour | wheat gluten | 1.33 | Sorbitol-70 | 0.32 | ULTRAMULSION ® | dried chicken | (5.0) |
| | (35.8) | (27) | | (16.7) | | (2) | (1.5) | |
| | | | | | | | Guar Gum | |
| | | | | | | | (12) | |
| 16 | Parsley Flour | wheat gluten | 0.07 | Sorbitol-Power | 0.40 | ULTRAMULSION ® | dried chicken | (5.0) |
| | (4) | (58.5) | | (25) | | (2) | (1.5) | |
| | Wheat bran | | | | | | | |
| | (4) | | | | | | | |
| 17 | Rice flour | wheat gluten | 0.5 | Sorbitol-70 | 0.17 | ULTRAMULSION ® | dried chicken | (5.0) |
| | (25) | (51) | | (13) | | (2) | (1.5) | |
| | | | | | | | Canola oil | |
| | | | | | | | (2.5) | |
| 18 | Rice flour | wheat gluten | 0.51 | Sorbitol-Power | 0.16 | ULTRAMULSION ® | dried | (5.0) |
| | (22) | (49) | | (13) | | (2) | chicken | |
| | Wheat bran | | | | | | (1.5) | |
| | (3) | | | | | | Canola oil | |
| | | | | | | | (4.5) | |
| 19 | Rice flour | wheat gluten | 0.4 | Sorbitol-Power | 0.22 | ULTRAMULSION ® | dried | (5.0) |
| | (20) | (54.5) | | (15) | | (2) | chicken | |
| | Parsley flour | | | | | | (1.5) | |
| | (2) | | | | | | | |
| 20 | Durham flour | wheat gluten | 10.5 | liquid fructose | 0.57 | MICRODENT ® | beef flavor | (4.7) |
| | (25.4) | (4.7) | | (28.85) | | (4) | (0.25) | |
| | tapioca starch | | | | | DCP | | |
| | (24.1) | | | | | (4) | | |
| | | | | | | TSPP | | |
| | | | | | | (4) | | |

To effectively remove, control and disrupt biofilms that are continuously forming on the teeth of pets requires that human intervention give way to pet self-administered biofilm therapy. Effective pet self-administration using the recovery pet chews of the present invention throughout the pet's waking hours is assured by relying on the following distinctive features of the recovery pet chews of the present invention:

Palatability: Generally the recovery pet chews of the present invention would be preferred over the various commercial pet products described in Table 1 in controlled palatability studies.

Chew-life, i.e., the time it takes to consume the chew. With a minimum of about a 4 minute chew-life, for the recovery pet chews of the invention, effective biofilm disruption, control and removal are assured, as well as effective delivery of a wide range of ingestible, therapeutic ingredients.

Shore-D Hardness: A Shore-D Hardness value approaching at least about 30, generally assures extended chew-life.

Physical Penetration: Values in the 600, and preferably 700 to 800 psi range, assure extended chew-life and sufficient chew elasticity to affect abrasive cleaning and biofilm removal during chewing.

Recovery Value: Quantifies the chew elasticity which has a major influence on chew-life; long term, consistent delivery of therapeutic ingredients; abrasive cleaning and removal of biofilms and tartar. Recover values of at least about 30% assure suitable chew-life, along with effective delivery of therapeutic ingredients, abrasion of tooth surfaces and control, disruption and removal of biofilms and tartar during chewing.

Certain of the foregoing features of the pet chews of the present invention, along with other properties, are detailed in Table 4 below.

The formulation detailed below was processed on commercial extruding/densifying equipment, such as detailed in U.S. Pat. No. 5,622,744. Extruded/densified recovery pet chews in the shape of round discs in varying thicknesses were produced and stored in plastic containers at room temperature. The discs were tested periodically. The results of these tests are reported in Table 4 below.

### EXAMPLE 21

Recovery Oral Care Pet Chew Commercial Production

A production operation in the manner of U.S. Pat. No. 5,622,744, was prepared consisting of a cooking extruder, Extru-Tech Model E525, feeding a conveyor belt leading to a product densification unit, Extru-Tech Model E750 (PDU). The cooking extruder was fitted with 7 screw segments. Segments 1 and 3 were uncut. Segments 4, 5, 6 and 7 were double flights with 2 inch triple cuts. The PDU was fitted with a 1.46 inch circular die. A rotary knife was set to cut the extruded rope into short cylinders. A batch of dry extruder feedstock was preconditioned by tumbling 430 lbs of durum flour, 412 lbs of tapioca starch, 74 lbs of wheat gluten, 15 lbs of dicalcium phosphate, 15 lbs of tetrasodium pyrophosphate and 30 lbs of ULTRAMULSION® (a solid emulsion of poloxamer 407 and polydimethylsiloxane in a weight ratio of 5 to 1). The feedstock batch was added into a conditioner over the cooking extruder. Water was injected into the throat of the cooking extruder at the rate of 44 lbs per hour. The addition rate of the preconditioned batch was 744 lbs per hour. A slurry of 77% liquid fructose, 30 lbs of water and 5 lbs of chicken flavor was pre-heated to 100° F. and injected into the throat of the cooking extruder at the rate of 374 lbs per hour. These production parameters were chosen to give a viscous rope of plasticized dough. The hot steaming dough was transported by conveyer belt from the extruder to the throat of the PDU. The PDU was set up with a rotary knife at 698 RPM. The PDU motor was controlled with a frequency drive and set at 28 Hz. Amperage was 45 AMP. PDU cooling jackets 1 to 4 were set as follows 1: off; 2: on; 3: on; 4: on. Pressure was measured at 375 PSI on the exit tube of the PDU. A densified dough rope was produced by the PDU and cooled to give a thick rope out of the die. The rotary knife cut cylinders from the rope to produce small disc shaped biscuits of varying thicknesses (typically 12 to ¾ inch thick). Drying at 95° F. for 22 minutes produced recovery pet chews with 0.59 available water.

TABLE 4
Recovery Pet Chew Attributes of Example 21
after Extrusion, Densification and Drying

| | | Available | | | | |
|---|---|---|---|---|---|---|
| | Chew | Water after | | Physical | | Approx. |
| Age of Chew | Thickness | drying | Density | Penetration | Shore - D | Chew-life |
| (in days) | (in inches) | (in %) | (gm/ml) | (in PSI) | Hardness | (in min) |
| 7 | ½ | .67 | 1.13 | 625 | 26 | 4-6 |
| 7 | ¾ | .67 | 1.21 | 350 | 27.5 | 4-6 |
| 14 | ½ | .62 | 1.13 | 600 | 31 | 4-6 |
| 14 | ¾ | .62 | 1.21 | 550 | 30.5 | 4-6 |
| 21 | ½ | .62 | 1.13 | 825 | 35 | 4-8 |
| 21 | ¾ | .62 | 1.21 | 750 | 33 | 4-8 |
| 28 | ½ | .59 | 1.13 | 825 | 32.5 | 4-8 |
| 28 | ¾ | .59 | 1.21 | 675 | 34.5 | 4-6 |
| 35 | ½ | .53 | 1.13 | 800 | 32 | 4-8 |
| 35 | ¾ | .53 | 1.21 | 775 | 35 | 4-8 |
| 42 | ½ | .59 | 1.13 | 775 | 32.5 | 4-8 |
| 42 | ¾ | .59 | 1.21 | 775 | 33 | 4-8 |
| 49 | ½ | n/a | 1.13 | 800 | 30 | 4-10 |
| 49 | ¾ | n/a | 1.21 | 775 | 33 | 4-10 |
| 56 | ½ | n/a | 1.13 | 775 | 32.5 | 4-10 |
| 56 | ¾ | n/a | 1.21 | 775 | 33 | 4-10 |
| 91 | ½ | n/a | 1.13 | 900 | 32.5 | 4-10 |
| 91 | ¾ | n/a | 1.21 | 850 | 34 | 4-10 |
| 240 | ½ | n/a | 1.13 | 1000 | 32 | 8-12 |
| 240 | ¾ | n/a | 1.21 | 1000 | 34 | 8-12 |

In addition to the specific: starches, proteins and plasticizing ingredients described in Examples 1 through 31, the following can be used in the recovery pet chews of the invention:

As to starches: Starches are present in the recovery pet chews of the invention at between about 7 and about 75 weight percent. The weight ratio of starch to protein in recovery pet chew blends of the invention is from between about 0.2 and about 5. Other suitable starches include: cornflower, hard wheat flour, soft wheat flour, oat flour, rice flour, potato flour, sorghum flour, amaranth flour, barley flour, tapioca starch, parsley flour, cassaya starch, yucca starch, durum wheat flour, buckwheat flour, sweet potato flour, millet flour, maize, rye, triticale, mung bean and mixtures thereof.

As to proteins: The following can also be used in the recovery pet chews of the invention: plant and animal proteins consisting of: oat gluten, wheat gluten, rice gluten, corn gluten, soy protein isolate, soy protein concentrate, potato protein, corn zein, hordein, avenin, kafirin, casein, whey, albumin collagen, gelatin, keratin, cow's milk, sheep's milk, goat's milk, chicken egg protein, fish meal, amaranth protein and mixtures thereof.

Protein is preferred in recovery pet chews of the invention at between about 3 and about 70 weight percent. The weight ratio of starch and protein to plasticizer in the recovery pet chew blends of the invention is from between about 5 and about 0.5.

As to plasticizers: The following can also be used in the recovery pet chews of the present invention: cane sugar, liquid fructose solutions, fructose solids, sucrose, glucose, mannose, maltose, glycerin, water, propylene glycol, sorbitol, honey, grape juice concentrate, corn syrup, molasses and mixtures thereof. Plasticizers are preferred in the recovery pet chews of the present invention at between about 3 and about 40 weight percent.

It has been discovered that, when the ingestible, recovery pet oral care chews of the present invention are extruded and densified with the emulsions such as MICRODENT® and/or ULTRAMULSION®, surprisingly, these emulsions are consistently releasable from the chew at biofilm disrupting and controlling levels, into the oral cavity during their chew-life. These emulsions can also contain various other ingredients, including flavorants, conditioners, mouthfeel agents, etc., which encourage the pet to continue to chew and help retain the pet's interest in masticating the entire recovery pet chew.

The melt emulsions described as MICRODENT® and ULTRAMULSION® are preferably contained substantially throughout the ingestible recovery pet chew of the invention. These emulsions are described in detail in the MICRODENT® and ULTRAMULSION® U.S. patents to Hill et al., referenced above. Generally, these melt emulsions comprise a coating agent emulsified in surfactants, such as:
sodium lauryl sulfate,
sodium lauryl sarcosinate,
polyethyleneglycol stearate,
polyethyleneglycol monostearate,
coconut monoglyceride sulfonates,
soap powder,
sodium alkyl sulfates,
sodium alkyl sulfoacetates,
alkyl polyglycol ether carboxylates such as those described in U.S. Pat. No. 4,130,636 polyoxyethylene derivatives or sorbitan esters, such as those described in U.S. Pat. No. 4,130,636, polyoxyethylene derivatives or sorbitan esters, such as those described in U.S. Pat. Nos. 3,639,563 and 3,947,570, propoxylated cetyl alcohol as described in U.S. Pat. No. 2,677,700, and
Preferred commercially available substances which include:
polyoxyethylene—polyoxypropylene block copolymers such as Pluronic F108, and F127 (BASF) and polysorbates such as Tween 40 and 80 (Hercules).
Particularly preferred surfactants include block copolymers comprising a congeneric mixture of conjugated polyoxypropylene and polyoxyethelene compounds having a hydrophobe, a polyoxypropylene polymer of at least 1200 molecular weight; such as described in U.S. Pat. Nos. 4,343,785; 4,465,663; 4,511,563 and 4,476,107.

Suitable coating substances for these melt emulsions can be functionally described as follows; they:
(1) suppress the tendency of the surfactant cleaners present to foam,
(2) are safely ingestible at the concentrations used,
(3) have an affinity for mouth and teeth surfaces,
(4) are neutral, inert and do not support biological activity,
(5) modify the surface energy properties of surfaces of the mouth such that it is more difficult for food particles, cellular debris and various plaque precursors and formers to attach to these surfaces, and
(6) form a thin, transparent, transient coating that does not build up on mouth surfaces and is removed by the normal cleaning and flushing action of the mouth.

Those coating substances suitable for the melt emulsions of the invention include various silicones, long chain hydrocarbons, carbowaxes and polymers, such as:
polydimethylsiloxanes at viscosities up to 25 million cs, with 2.5 million cs preferred,
long chain hydrocarbons, especially normal paraffins having a chain length of 16 carbon atoms or greater, paraffins with several loci of branching and unsaturation does not create unacceptable toxicity nor lower the solidification point below body temperature,
Carbowaxes® (polyethylene glycols) and polymers which have limited solubility in ethanol and water solutions where the ethanol or water ratio is greater than 0.3:1 but have essentially no solubility in water or saliva at lower ratios.

Those conditioners suitable for inclusion in the recovery chew extruding and densifying process of the present invention are preferably selected primarily from several classes of high molecular weight substances, such as:
Purified, soluble proteins such as sodium caseinate, various cereal glutens, albumins and the like,
Starches and modified starches,
Soluble cellulose derivatives such as carboxymethylcellulose, hydroxymethyl cellulose, and hydroxypropylcellulose,
Polyhydroxyalcohols such as hydrogenated glucose syrup,
Polyethylene and polypropylene glycols, and
Water soluble resins such as Gantrez®.

In addition, low molecular weight polyols such as glycerin and sorbitol and other humectants may also serve as conditioners, either in combination with high molecular weight substances such as discussed above or alone.

In addition to MICRODENT® and/or ULTRAMULSION®, various other therapeutic substances that are soluble or dispersible in said emulsions can be contained throughout the recovery pet chew of the invention. These therapeutic ingredients include:
toothpaste ingredients including anti-tartar ingredients including: sodium hexametaphosphate, tetrasodium pyrophosphate, various other pyrophosphates and sequestering agents, etc.,
whitening ingredients such as calcium peroxide, magnesium peroxide, carbamide peroxide, etc.,
cationic antimicrobials such as chlorhexidine diacetate, chlorhexidine digluconate, cetylpyridinium chloride, domiphen bromide, benzalkonium chloride, benzethonium chloride, and alexidine,
other antimicrobials such as triclosan and metronidazole
anti-caries ingredients including: sodium fluoride, fluorohexametaphosphate, stannous fluoride, etc.,
antibiotics, antiseptics, coagulants, vitamins, nutraceuticals, etc.,
flavorants and mouth conditioners,
cleaners including: cationic, anionic, nonionic and amphoteric surfactants, and
abrasives including: silica, pumice, dicalcium phosphate (DCP), tetrasodium pyrophosphate (TSPP).

Other therapeutic oral care ingredients are described in Examples 22 through 28 in Table 5 and include:
(a) substances that help reduce dental plaque acids and help inhibit the growth of s. mutans such as xylitol, lactitol, hydrogenated starch hydrolyzate (HSH), and
(b) substances associated with oral health including: baking soda, zinc compounds such as zinc citrate, coenzyme Q₁₀, folic acid, aloe vera, green tea, blood root, birch bark, grape seed extract, etc.

Additional pet therapeutic ingredients are described in Examples 29 through 31 in Table 6 and include:
(a) minerals including: calcium, potassium, magnesium and sodium; trace minerals including: iron, phosphorous, zinc, manganese, iodine, selenium and cobalt; dietary fibers including: wheat bran, soluble fibers such as from vegetables, fruits and grains such as oats, barley, etc.;
(b) nutraceuticals including: Echinacea purpurea, glucosamine, calcium, St. John's Wort, ginseng, riboflavin, biotin, folic acid, thiamine, panthothermic acid, chondroitin, hyaluronic acid, flaxseed, omega fatty acids, various enzyme, etc.;
(c) vitamins including: Vitamins: A, B, C, E, etc.; and
(d) pharmaceuticals including: parasite control ingredients such as: ivermectin, pyrantel, moxydectin, lufenuron, nitenpyram, NSAIDs such as Cosequin DS.

Further, the flavor/conditioner/mouthfeel agent/emulsion contained in the recovery pet chews of the present invention are more effective in physically cleaning pet tooth surfaces due to the “drive-to-chew” attributed to these substances distributed substantially throughout the recovery pet chew. This intense “drive-to-chew” prompted by the flavorant/conditioner/mouthfeel agent dispersed throughout the recovery chew results in more vigorous chewing and gnawing, by the pet than is normally associated with pet chews.

In addition to including flavorants, conditioners, etc., and/or therapeutic ingredients in the emulsions, the present invention also includes the addition of other ingredients such as dispersible abrasives into these emulsions. The distribution substantially throughout the recovery chew of an emulsion of MICRODENT® and/or ULTRAMULSION® containing anti-tartar ingredients and also containing, dispersed therein, various abrasives of various particle sizes, provides an in-situ-type toothpaste formulation that is continuously worked over tooth surfaces as the MICRODENT® and/or ULTRAMULSION® with abrasives dispersed therein is released from the recovery chew over its chew-life.

The illustrative Examples 22 through 31 as set forth in Tables 5 and 6 below teach the addition of a wide range of other therapeutic ingredients into the base compositions of suitable recovery pet chews of the invention.

TABLE 5
Examples 22 through 28 of additional Recovery Oral Care Pet Chew Formulations

| | | | | Oral Care | Other | |
|---|---|---|---|---|---|---|
| | | | | Emulsions | therapeutic | Total |
| | Starch | Protein(s) | Plasticizing Agent | (% by wt./viscosity of | Ingredients | Water |
| Ex. No. | (% by wt.) | (% by wt.) | (% by wt.) | silicone in cs) | (% by wt.) | (% by wt.) |
| 22 | Corn flour (12) | Oat gluten (1) | Sugar cane (12) | MICRODENT ® | TSPP (5) | 18 |
| | Hard wheat flour (12) | Wheat gluten (2) | Liquid fructose (12) | (5/1000) | DCP (2) | |
| | Soft wheat flour (12) | Chicken egg protein (1) | | | Xylitol (4) | |
| | | Fish meal (2) | | | | |
| 23 | Rice flour (14) | Rice gluten (1) | Sucrose (14) | MICRODENT ® | Silica (5) | 16 |
| | Potato flour (8) | Corn gluten (1) | Mannose (12.5) | (6/1500) | Zinc concentrate (0.5) | |
| | Sorghum flour (16) | Soy protein isolate (2) | | | Echinacea purpurea (2) | |
| | | Amaranth protein (2) | | | | |
| 24 | Amaranth flour (9) | Soy protein concentrate (4) | Maltose (20) | MICRODENT ® | Vitamins C & E (0.2) | 17 |
| | Barley flour (8) | Potato protein (1) | Glycerin (9) | (7/20,000) | Iodine (0.002) | |
| | Tapioca starch (17) | Corn zein (1) | | | Co-enzyme Q₁₀ (0.001) | |
| | Parsley flour (7) | | | | | |
| 25 | Cassava starch (8) | Hordein (2) | Propylene glycol (9) | MICRODENT ® | Grape seed extract (0.5) | 14.5 |
| | Yucca starch (8) | Avenin (1) | Sorbitol (20) | (6/15,000) | Lactitol (2) | |
| | Durum wheat flour (26) | Kafirin (3) | | | | |
| 27 | Buck wheat flour (26) | Casein (1) | Honey (18) | ULTRAMULSION ® | HSH (0.5) | 15.5 |
| | Sweet potato flour (9) | Whey (4) | Grape juice | (4/6 million) | Folic acid (0.001) | |
| | Millet flour (9) | Albumin collagen (1) | concentrate (9) | | Iron (0.001) | |
| | | Gelatin (2) | | | Wheat bran (1) | |
| 28 | Maize (17) | Keratin (2) | Corn syrup (7) | ULTRAMULSION ® | Selenium (0.001) | 15.5 |
| | Rye flour (9) | Cow's milk (3) | Molasses (20) | (6/2.5 million) | Vitamins A&B (0.001) | |
| | Triticale (10) | Sheep's milk (1) | | | Aloe vera (0.5) | |
| | Mung bean (8) | Goat's milk (1) | | | | |

TABLE 6
Examples 29 through 31 of additional Recovery Therapeutic Pet Chew Formulations

| | | | Plasticizing | Emulsions | Therapeutic | Added |
|---|---|---|---|---|---|---|
| Ex. | Starch | Protein(s) | Agent | (% by wt./viscosity of | Ingredients | Water |
| No. | (% by wt.) | (% by wt.) | (% by wt.) | silicone in cs) | (% by wt.) | (% by wt.) |
| 29 | Tapioca Starch | Wheat Gluten | Liq. Fructose | Microdent ® | Carprofen (0.04) | 3.4 |
| | (27.06) | (4.9) | (30.4) | (2/1000) | TSPP(2) | |
| | Wheat flour | | | | DCP(2) | |
| | (28.2) | | | | | |
| 30 | Tapioca Starch | Wheat Gluten | Liq. Fructose | Microdent ® | Carprofen (0.08) | 3.4 |
| | (27.02) | (4.9) | (30.4) | (2/1000) | TSPP (2) | |
| | Wheat Flour | | | | DCP (2) | |
| | (28.2) | | | | | |
| 31 | Tapioca Starch | Wheat Gluten | Liq. Fructose | Microdent ® | Glucosamine (2.7) | 3.3 |
| | (26.25) | (4.9) | (29.5) | (2/1000) | TSPP(2), | |
| | Wheat Flour | | | | DCP (2) | |
| | (27.3) | | | | Chondroitin (0.05) | |

The illustrative examples 29 through 31 as set forth in Table 6 above describe recovery pet chews used for purposes other than pet oral care, including introducing medicaments, vitamins and nutraceuticals into the pet's mouth.

The foregoing Examples illustrate preferred methods for manufacturing the pet chews of the present invention, as well as various preferred compositions for pet chews of the invention. Other methods including injection molding and comparable compositions would be suitable and would be obvious to one skilled in the art, following the teachings of this specification.

What is claimed is:

1. An ingestible, recovery pet chew comprising a blend of emulsion, starch, protein, water and a plasticizing substance having a recovery value of at least about 30%.

2. A recovery pet chew according to claim 1, wherein the weight ratio of said starch to said protein is from between about 0.1 and about 10 and said emulsion is present at at least about 3% by weight.

3. A recovery pet chew according to claim 2, wherein said plasticizing substance is selected from the group consisting of fructose, cane syrup, honey, grape juice concentrate, corn syrup, molasses, sorbitol, sucrose and glucose, mannose, maltose, glyceria, propylene glycol, glycerin and mixtures thereof.

4. A recovery pet chew according to claim 2, wherein said protein is selected from the group consisting of oat gluten, wheat gluten, rice gluten, corn gluten, soy protein isolate, soy protein concentrate, potato protein, corn zein, hordein, avenin, kafirin, casein, whey, albumin collagen, gelatin, keratin, cow's milk, sheep's milk, goat's, milk, chicken egg protein, fish meal, amaranth protein and mixtures thereof.

5. A recovery pet chew according to claim 2, wherein said starch is selected from the group consisting of cornflower, hard wheat flour, soft wheat flour, oat flour, rice flour, potato flour, sorghum flour, amaranth flour, barley flour, tapioca starch, parsley flour, cassaya starch, yucca starch, durum wheat flour, buckwheat flour, sweet potato flour, millet flour, maize, rye, triticale, mung bean and mixtures thereof.

6. A recovery pet chew according to claim 2, wherein the weight ratio of said starch and protein to said plasticizer is from between about 5 and about 0.5.

7. A recovery pet chew according to claim 2, wherein said available water is present at between about 0.4 and about 0.7% by weight.

8. A pet chew according to claim 1, wherein said chew is processed using extruding and densifying means.

9. A recovery pet chew according to claim 1, wherein said pet chew indicates:
(a) physical penetration between about 200 and about 1000 psi,
(b) a Shore-D Hardness of at least about 25,
(c) recovery value of at least about 30%, and
(d) a chew-life of at least about 4 minutes.

10. A recovery pet chew according to claim 1, having a density greater than about 1.1.

11. An oral care, ingestible, recovery, oral care pet chew comprising: a blend of an emulsion, starch, protein, water, a plasticizing substance and an oral care ingredient.

12. A recovery oral care pet chew according to claim 11, wherein said oral care ingredient is an antimicrobial disrupting and controlling emulsion.

13. A recovery oral care pet chew according to claim 12, wherein said antimicrobial is selected from the group consisting of cetylpyridinium chloride, triclosan, chlorhexidine digluconate, domiphen bromide, benzalkonium chloride, benzethonium chloride, alexidine and mixtures thereof.

14. A recovery oral care pet chew according to claim 11, wherein the weight ratio of said starch to said protein is from between about 0.1 and about 10.

15. A recovery oral care pet chew according to claim 11, wherein said plasticizing substance is selected from the group consisting of fructose, cane syrup, honey, grape juice concentrate, corn syrup, molasses, sorbitol, sucrose and glucose, mannose, maltose, glyceria, propylene glycol, glycerin and mixtures thereof.

16. A recovery oral care pet chew according to claim 11, wherein said protein is selected from the group consisting of oat gluten, wheat gluten, rice gluten, corn gluten, soy protein isolate, soy protein concentrate, potato protein, corn zein, hordein, avenin, kafirin, casein, whey, albumin collagen, gelatin, keratin, cow's milk, sheep's milk, goat's, milk, chicken egg protein, fish meal, amaranth protein and mixtures thereof.

17. A recovery oral care pet chew according to claim 11, wherein said starch is selected from the group consisting of cornflower, hard wheat flour, soft wheat flour, oat flour, rice flour, potato flour, sorghum flour, amaranth flour, barley flour, tapioca starch, parsley flour, cassaya starch, yucca starch, durum wheat flour, buckwheat flour, sweet potato flour, millet flour, maize, rye, triticale, mung bean and mixtures thereof.

18. A recovery oral care pet chew according to claim 11, wherein the weight ratio of said starch and protein to said plasticizer is from between about 5 and about 0.5.

19. A recovery oral care pet chew according to claim 11, wherein said available water is present at between about 0.4 and about 0.7% by weight.

20. A recovery oral care pet chew according to claim 11, wherein said chew is processed using extruding and densifying means.

21. A recovery oral care pet chew according to claim 11, wherein said chew has
(a) a physical penetration between about 200 and about 1000 psi,
(b) a Shore-D Hardness of at least about 25,
(c) a recovery value of at least about 30%, and
(d) a chew-life of at least about 4 minutes.

22. A recovery oral care pet chew according to claim 11, having a density greater than about 1.1.

23. A method for controlling, disrupting and removing biofilms from pet's teeth, comprising periodically feeding said pet an ingestible, recovery, pet oral care chew, comprising: a blend of an emulsion, starch, protein, a plasticizer and water, wherein said chew has:
(a) a physical penetration between about 200 and about 1000 psi;
(b) a Shore-D Hardness of at least about 25,
(c) a chew-life of at least about 4 minutes; and
(d) a recovery value of at least about 30%.

24. A method for manufacturing an oral care, ingestible, recovery pet chews, comprising: extruding and densifying a blend comprising: an emulsion, starch, protein, water and a plasticizer, wherein said extruded, densified chew has:
(a) a physical penetration between about 200 and 1000 psi;
(b) a chew-life of at least about 4 minutes;
(c) an estimated recovery value of at least about 30%;
(d) a density greater than about 1.1; and
(e) a Shore-D Hardness of at least about 25.

25. An recovery oral care pet chew according to claim 11 containing oral care abrasives selected from the group consisting of silica, pumice, trisodiumphosphate, tetrasodiumpyrophosphate, dicalciumphosphate and mixtures thereof.

26. A recovery oral care pet chew according to claim 11 containing additional oral care surfactants selected from the group of surfactants consisting of nonionic, anionic, cationic, amphoteric and mixtures thereof.

27. A recovery oral care pet chew according to claim 1 containing oral care antimicrobials selected from the group consisting of cetylpyridinium chloride, triclosan, chlorhexidine digluconate, domiphen bromide, benzalkonium chloride, benzethonium chloride, alexidine and mixtures thereof.

28. A biofilm therapy, recovery pet chew suitable for controlling, disrupting and removing biofilms from the surfaces of pet teeth comprising: a blend of an emulsion, starch, protein, a plasticizer and water having:
(a) a physical penetration between about 200 and about 1000 psi;
(b) a Shore-D Hardness of at least about 25;
(c) a chew-life of at least about 4 minutes; and
(d) a recovery value of at least about 30%.

29. A method for manufacturing oral care, ingestible, recovery pet chews, comprising extruding and densifying a blend comprising: starch, protein, a plasticizer and water, wherein said extrudate has:
(a) a physical penetration between about 200 and about 1000 psi;
(b) a chew-life of at least about 4 minutes;
(c) an estimated recovery value of at least about 30%;
(d) a density greater than about 1.1; and
(e) a Shore-D Hardness of at least about 25.

30. A therapeutic, extruded and densified, ingestible, recovery pet chew, comprising: a blend of an emulsion, starch, protein, water, a plasticizing substance and a pet therapeutic ingredient.

31. A therapeutic, recovery pet chew according to claim 30, wherein said pet therapeutic ingredient is selected from the group consisting of vitamins, minerals, pharmaceuticals, nutraceuticals, systemic treatment substances and mixtures thereof.

32. A therapeutic, recovery pet chew according to claim 30, wherein the weight ratio of said starch to said protein is from between about 0.1 and about 10.

33. A therapeutic, recovery pet chew according to claim 30, wherein said plasticizing substance is selected from the group consisting of fructose, cane syrup, honey, grape juice concentrate, corn syrup, molasses, sorbitol, sucrose and glucose, mannose, maltose, glyceria, propylene glycol, glycerin and mixtures thereof.

34. A therapeutic, recovery pet chew according to claim 30, wherein said protein is selected from the group consisting of oat gluten, wheat gluten, rice gluten, corn gluten, soy protein isolate, soy protein concentrate, potato protein, corn zein, hordein, avenin, kafirin, casein, whey, albumin collagen, gelatin, keratin, cow's milk, sheep's milk, goat's, milk, chicken egg protein, fish meal, amaranth protein and mixtures thereof.

35. A therapeutic, recovery pet chew according to claim 30, wherein said starch is selected from the group consisting of cornflower, hard wheat flour, soft wheat flour, oat flour, rice flour, potato flour, sorghum flour, amaranth flour, barley flour, tapioca starch, parsley flour, cassaya starch, yucca starch, durum wheat flour, buckwheat flour, sweet potato flour, millet flour, maize, rye, triticale, mung bean and mixtures thereof.

36. A therapeutic, recovery pet chew according to claim 30, wherein the weight ratio of said starch and protein to said plasticizer is from between about 5 and about 0.5.

37. A therapeutic, recovery pet chew according to claim 30, wherein said available water is present at between about 0.4 and about 0.7% by weight.

38. A therapeutic, recovery pet chew according to claim 30, wherein the extrudate is subsequently injection molded.

39. A therapeutic, recovery pet chew according to claim 30, wherein said chew has:
(a) a physical penetration between about 200 and about 1000 psi,
(b) a recovery value of at least about 30%; and
(c) a chew-life of at least about 4 minutes.

40. A therapeutic, recovery pet chew according to claim 30, wherein the density is greater than about 1.1.

41. A method for manufacturing therapeutic, ingestible, recovery, therapeutic pet chews comprising: extruding and densifying a blend comprising: an emulsion, starch, protein, water, a plasticizer and a therapeutic ingredient, wherein said extruded, densified chew indicates:
(a) physical penetration between about 200 and 1000 psi;
(b) a chew-life of at least about 4 minutes;
(c) an estimated recovery value of at least about 30%;
(d) a density greater than about 1.1; and
(e) a Shore-D Hardness of at least about 25.
